(19) United States
(12) Patent Application Publication
(10) Pub. No.: US 2023/0380679 A1
(43) Pub. Date: Nov. 30, 2023

(54) SYSTEMS, METHODS, AND DEVICES FOR VISION ASSESSMENT AND THERAPY

(71) Applicant: Vivid Vision, Inc., San Francisco, CA (US)

(72) Inventors: Benjamin Backus, San Francisco, CA (US); Brian Dornbos, San Francisco, CA (US); Tuan Tran, San Francisco, CA (US); James J. Blaha, San Francisco, CA (US); Manish Gupta, San Francisco, CA (US)

(21) Appl. No.: 18/450,227

(22) Filed: Aug. 15, 2023

Related U.S. Application Data
(63) Continuation of application No. PCT/US2022/018388, filed on Mar. 1, 2022.
(60) Provisional application No. 63/215,182, filed on Jun. 25, 2021.
(60) Provisional application No. 63/155,720, filed on Mar. 2, 2021.
(60) Provisional application No. 63/155,141, filed on Mar. 1, 2021.

(51) Int. Cl. A61B 3/10; A61B 3/02; G16H 50/20; G16H 20/30

(52) U.S. Cl. CPC A61B 3/10; A61B 3/02; G16H 50/20; G16H 20/30

(57) ABSTRACT

A computer system is provided. The system includes a computing device including at least one data processor and at least one computer readable storage medium storing computer-executable instructions. The system further includes a device configured to communicate with the computing device and having a display. The at least one data processor is configured to execute the computer executable instructions to perform operations. The operations including displaying, using the at least one data processor and on the display, a first eye condition assessment or eye condition correction activity of a plurality of eye condition assessments or correction activities configured to measure one or more visual skills of a user. The operations further include receiving, by the at least one data processor, user input with respect to the first eye condition assessment or eye condition correction activity. The operations further include determining whether a target value of at least one parameter has been reached.

### CROSS-REFERENCE TO RELATED APPLICATIONS

This application claims priority to co-pending U.S. Provisional Application Ser. No. 63/155,141, filed Mar. 1, 2021, 63/155,720, filed Mar. 2, 2021, and 63/215,182, filed Jun. 25, 2021. The entire contents of each are hereby incorporated by reference herein in their entireties.

### TECHNICAL FIELD

The subject matter described herein relates to a vision assessment and correction system including an automated customized treatment for health disorders using a series of feedback loops where the end-user patient performs a test or series of tests that drive treatment activities.

### BACKGROUND

Many people suffer from various vision disorders that are often left undiagnosed and untreated. Some visual problems affect a person since childhood, and, if not detected and treated in a timely manner, can result in a permanent loss of vision as the person gets older. For example, amblyopia, or “lazy eye,” is a common visual disorder afflicting approximately 4% of the population in the United States. Amblyopia results from an incompatibility of visual perception between the brain and the amblyopic, “weak” eye, such that the other, “strong” eye, inhibits the amblyopic eye which results in a permanent decrease in vision in that eye. Amblyopia typically occurs in children, but adult cases occur as well.

A typical treatment for amblyopia involves the subject's wearing an eye patch over the unaffected eye with the goal of forcing the person to use the weaker eye to thus train that eye to become stronger. However, patients, particularly children, tend to view such treatment as inconvenient and uncomfortable, which results in poor compliance and therefore leads to unreliable results. Measuring a progress of such treatment can be challenging. Furthermore, a detection of amblyopia and other vision disorders in young children can be complicated.

### SUMMARY

As discussed in greater detail below, features of the current subject matter can enable automated customized treatment for vision disorders. Features of the current subject matter may support a vision assessment and correction system and methods.

In one aspect, a computer system for vision assessment and correction is provided. The system includes a computing device including at least one data processor and at least one computer readable storage medium storing computer-executable instructions. The system further includes a device configured to communicate with the computing device and having a display configured to interact with the user having a medical disorder or perceptual skill. The at least one data processor is configured to execute the computer executable instructions to perform operations. The operations including displaying, using the at least one data processor and on the display, a first eye condition assessment or eye condition correction activity of a plurality of eye condition assessments or eye condition correction activities configured to measure one or more visual skills of a user. The operations further include receiving, by the at least one data processor, user input with respect to the first assessment or correction activity. The operations further include determining, by the at least one data processor using the received user input, whether a target value of at least one parameter has been reached. The target value of the at least one parameter is indicative of a perception of a first property of the first eye condition assessment or eye condition correction activity by at least one eye of the user and based on the target parameter being reached, determining a particular state of the user out of a plurality of possible states within a model of the user's medical disorder or perceptual skill that the user hopes to improve. The operations further include when it is determined that the target value has changed, using the at least one data processor, updating eye conditions assessments and/or correction activities scheduled for the user by the computing device to reflect the user's current state within the model of the user's medical disorder. The operations further include iteratively performing the displaying, receiving, determining, and updating steps until it is determined that the user's medical condition or perceptual skill has improved to the point where the target value has been reached.

Implementations of the current subject matter can include methods consistent with the descriptions provided herein as well as articles that comprise a tangibly embodied machine-readable medium operable to cause one or more machines (e.g., computers, etc.) to perform operations implementing one or more of the described features. Similarly, computer systems are also described that may include one or more processors and one or more memories coupled to the one or more processors. A memory, which can include a computer-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

In another aspect, a computer system for vision assessment and correction is provided. The system includes at least one data processor configured to perform operations including: displaying, using the at least one programmable processor and on a display, a first eye condition assessment or eye condition correction activity of a plurality of eye condition assessments or eye condition correction activities configured to measure a medical disorder or perceptual skill of a user viewing the display; receiving, by the at least one programmable processor, user input with respect to the first assessment or correction activity, the user input being generated based on input acquired from the user during the eye condition assessment or eye condition correction activity; determining, by the at least one programmable processor using the received user input, whether a target value of at least one parameter has been reached, wherein the target value of the at least one parameter is indicative of a perception of the first property of the first eye condition assessment or eye condition correction activity by at least one eye of the user; determining, by the at least one programmable processor based on the target parameter being reached, a current state of the user out of a plurality of possible states within a model of the medical disorder or perceptual skill of the user; when it is determined that the target value has changed, updating, by the at least one programmable processor, eye conditions assessments and/or eye condition correction activities scheduled for the user by the computing device to reflect the user's current state within the model of the user's medical disorder; and iteratively performing, by the at least one programmable processor, the displaying, receiving, determining, and updating steps until it is determined that the user's medical condition or perceptual skill has improved to the point where the target value has been reached.

In another aspect, a method is provided, the method including: displaying a first eye condition assessment or eye condition correction activity of a plurality of eye condition assessments or eye condition correction activities configured to measure one or more visual skills of a user; receiving user input with respect to the first assessment or correction activity, the user input being generated based on input acquired from the user during the eye condition assessment or eye condition correction activity; determining whether a target value of at least one parameter has been reached, wherein the target value of the at least one parameter is indicative of a perception of a first property of the first eye condition assessment or eye condition correction activity by at least one eye of the user; determining, based on the target parameter being reached, a current state of the user out of a plurality of possible states within a model of a medical disorder or perceptual skill of the user; updating, when it is determined that the target value has changed, eye conditions assessments or correction activities scheduled for the user to reflect the user's current state within the model of the medical disorder or perceptual skill of the user; and iteratively performing the displaying, receiving, determining, and updating steps until it is determined that the medical condition or perceptual skill of the user has improved to the point where the target value has been reached.

In another aspect, a computer program product is provided, including a non-transitory computer readable medium storing instructions that, when executed by at least one programmable processor, result in operations. The operations include: displaying a first eye condition assessment or eye condition correction activity of a plurality of eye condition assessments or eye condition correction activities configured to measure one or more visual skills of a user; receiving user input with respect to the first assessment or correction activity, the user input being generated based on input acquired from the user during the eye condition assessment or eye condition correction activity; determining whether a target value of at least one parameter has been reached, wherein the target value of the at least one parameter is indicative of a perception of a first property of the first eye condition assessment or eye condition correction activity by at least one eye of the user; determining, based on the target parameter being reached, a current state of the user out of a plurality of possible states within a model of a medical disorder or perceptual skill of the user; updating, when it is determined that the target value has changed, eye conditions assessments or correction activities scheduled for the user to reflect the user's current state within the model of the medical disorder or perceptual skill of the user; and iteratively performing the displaying, receiving, determining, and updating steps until it is determined that the medical condition or perceptual skill of the user has improved to the point where the target value has been reached.

In implementations, the following features may be included individually or in combination. The medical disorder or perceptual skill may include at least one of anisometropic amblyopia, strabismic amblyopia, Intermittent exotropia, constant exotropia, a disorder of vergence, convergence insufficiency, convergence excess, divergence insufficiency, divergence excess, esotropia—intermittent, esotropia, hypertropia, cortical suppression, lack of sensory binocularity needed for flat fusion, lack of sensory binocularity from vergence insufficiency, accommodative insufficiency, accommodative infacility, traumatic brain injury (TBI), anomalous correspondence, oculomotor, glaucoma, low-vision training, brain injury, visual field loss, neglect, confusion, scotoma, lack of or impaired stereoscopic depth perception, a sports vision-related condition. The plurality of eye condition assessments or eye condition correction activities may include one or more of: vergence range; visual acuity; contrast sensitivity; central-peripheral awareness; stereo depth; visual processing; perceptual; reaction time; accommodation; tracking accuracy; flat fusion; stereopsis; awareness of diplopia; and minimized fixation disparity. The device may include one or more of a virtual reality device, a personal computer, a smartphones, an augmented reality device, a mixed reality device, an eye tracking device, a wearable display, a lens, a body movement tracking device, a facial tracking device, an appendage tracking device, a haptic feedback device, an audible sensor, a microphones, and/or a biosensor.

The computing system may further include a device configured to communicate with the at least one programmable processor, the device including a display. The displaying may be done using a device having a display configured to interact with the user. The displaying may occur using a device having a display configured to communicate with the user. The displaying, the receiving, the determining, the updating, and the iteratively performing may be performed using one or more programmable processors.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to an enterprise software system or other content management software solution or architecture, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations.

In the drawings,

FIG. 1 shows a flowchart illustrating a process of operating a computing system to assess and/or treat a vision disorder of a person;

FIG. 2 is a block diagram illustrating a computer system in which the process of FIG. 1 can be implemented, consistent with implementations of the current subject matter;

FIG. 3 is a flowchart illustrating a process of operating a computing system to assess and/or treat a vision disorder of a person;

FIG. 4 depicts a block diagram of an example computing apparatus, in accordance with some example implementations;

FIG. 5 depicts a diagram for an example treatment process for an example vision disorder; and

FIG. 6 illustrates a flowchart for vision assessment and correction therapy, in accordance with some example implementations.

FIG. 7 illustrates another flowchart for vision assessment and correction therapy, in accordance with some example implementations.

FIG. 8 illustrates another flowchart for vision assessment and correction therapy, in accordance with some example implementations.

FIG. 9 illustrates another flowchart for vision assessment and correction therapy, in accordance with some example implementations.

FIG. 10 illustrates an example screen of a user interface for a system of vision assessment and correction therapy, in accordance with some example implementations.

FIG. 11 illustrates another example screen of a user interface for a system of vision assessment and correction therapy, in accordance with some example implementations.

FIG. 12 illustrates another example screen of a user interface for a system of vision assessment and correction therapy, in accordance with some example implementations.

FIGS. 13A-13B illustrate an example patient dashboard screen (FIG. 13A) and example progress wheels (FIG. 13B) of user interface for a system of vision assessment and correction therapy, in accordance with some example implementations.

FIG. 14 illustrates an example of vision assessment task completion motivation scheme in accordance with some example implementations.

FIG. 15 illustrates another example screen of a user interface for a system of vision assessment and correction therapy, in accordance with some example implementations.

FIG. 16 illustrates another example screen of a user interface for a system of vision assessment and correction therapy, in accordance with some example implementations.

FIG. 17 illustrates another example screen of a user interface for a system of vision assessment and correction therapy, in accordance with some example implementations.

FIG. 18 shows a chart of underlying vision skills that may be tested using the systems and methods described herein, including examples.

FIG. 19 illustrates another example screen of a user interface for a system of vision assessment and correction therapy, in accordance with some example implementations.

FIG. 20 illustrates another example screen of a user interface for a system of vision assessment and correction therapy, in accordance with some example implementations.

FIG. 21 illustrates another example screen of a user interface for a system of vision assessment and correction therapy, in accordance with some example implementations.

FIG. 22 illustrates another example screen of a user interface for a system of vision assessment and correction therapy, in accordance with some example implementations.

FIG. 23 illustrates another example screen of a user interface for a system of vision assessment and correction therapy, in accordance with some example implementations.

FIG. 24 illustrates another example screen of a user interface for a system of vision assessment and correction therapy, in accordance with some example implementations.

FIG. 25 illustrates another example screen of a user interface for a system of vision assessment and correction therapy, in accordance with some example implementations.

FIG. 26 is a flowchart of the vision assessment and correction therapy system of FIG. 7.

FIG. 27 depicts a diagram for an example treatment process for an example vision disorder.

### DETAILED DESCRIPTION

Certain exemplary aspects of the current subject matter will now be described to provide an overall understanding of the principles of the systems and methods disclosed herein. One or more examples of these aspects are illustrated in the accompanying drawings. Those skilled in the art will understand that the systems and methods specifically described herein and illustrated in the accompanying drawings are non-limiting exemplary aspects and that the scope of the aspects is defined solely by the claims. Further, the features illustrated or described in connection with one exemplary aspect may be combined with the features of other aspects. Such modifications and variations are intended to be included within the scope of the described subject matter.

The current subject matter provides methods, systems, and computer program products to detect, assess and treat vision disorders in subjects. The system can include a computing device configured to communicate with a device (e.g., a head-mountable virtual reality (VR)) that creates a virtual reality environment for a user wearing the device such that a display, or screen, is positioned over the user's eyes. The computing device and/or the device may include at least one data processor, a visual interface, and memory storing instructions for execution by the at least one data processor.

The current subject matter can be used to address binocular vision disorders (e.g., amblyopia, lack of stereo, strabismus, and/or vergence disorders). It can also be used to treat accommodative disorders, or more generally, disorders of the visual system or body. The use of eye assessment and/or training systems can, in some implementations, require vigilance and expertise on the part of the clinician, and as a result, treatments may not always be optimized for the patient. For example, clinicians may be unsure what settings to use or how settings should be adjusted during treatment and training activities when they use the product; similarly, a clinician may fail to adjust or alter treatment to continually challenge a patient's visual ability (or abilities), resulting in a treatment plateau. The vision assessment and correction system and associated systems, methods, devices, software, and/or algorithms described herein can be beneficial as a guide for treatment whether it occurs in the clinic or at home.

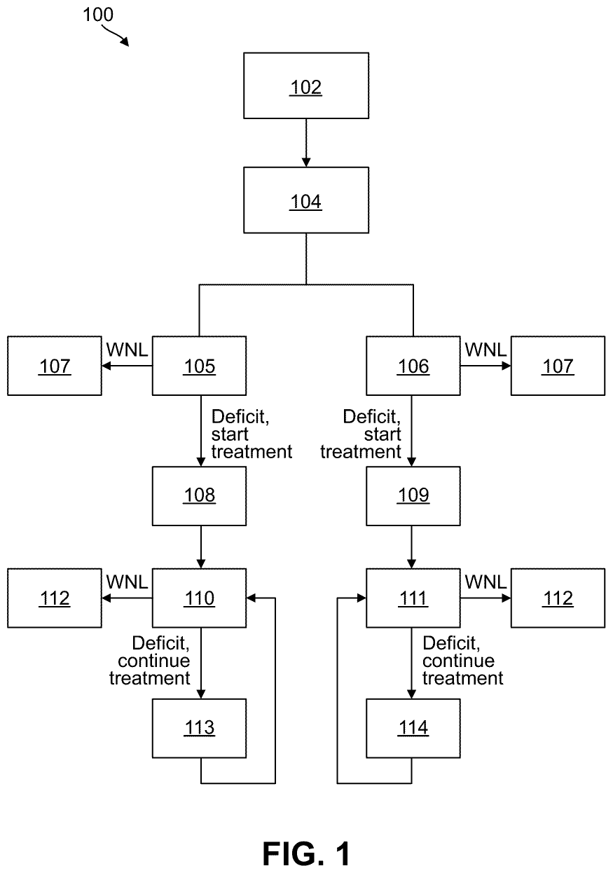

FIG. 1 is a process flow diagram 100 illustrating a method of operating a computing system to assess and/or treat a vision disorder of a person. The computing system can include at least one data processor and memory communicatively coupled to the at least data processor. The memory can be configured to store computer-executable instructions embodied as a vision correction platform or application. The computer-executable instructions, when executed by the at least one data processor, perform the described method. Furthermore, in some aspects, the entire or part of the platform can be stored on a remote computing device and can be accessible via the computing device. The computing system is in communication with a head-mountable display (e.g., a virtual reality (VR), augmented reality (AR), or the like), as discussed in more detail below.

As shown in FIG. 1, the process 100 can start at block 102, at any suitable time. At the block 102, a condition or an issue in a user or patient's vision can be identified by one or more of perception by the user, identification by a clinician administering a manual test, or automated identification by the user or the clinician. The process 100 may continue at block 104 where the computing system may assess the user or patient's vision. For example, the user may, at block 105, perform a first skill test or, at block 106, a second skill test to assess the user's vision. If the user can perform the skill (e.g., the first skill test or the second skill test) within normal limits (WNL), then at block 107, no further treatment may be necessary, and the system can proceed to testing another skill (e.g., the other of the first skill test or the second skill test, a third skill test, a skill N test, etc.). However, if the user cannot perform or pass the test (e.g., first skill test) to a sufficient level, then the system may proceed to block 108, and implement an automated track for treatment of a disorder or impairment identified/determined in the initial test (e.g., a disorder or impairment associated with a corresponding one of the first skill or the second skill at block 105 or block 106). If the user cannot perform the second skill test, at block 109, the system may implement an automated track for treatment of the disorder or impairment identified/determined in the initial test. In some implementations, the specified skill (e.g., the first skill test or the second skill test) can again be tested (e.g., at block 110 for the first skill test or block 111 for the second skill test, respectively) to generate a base line value for comparison to further (downstream) testing of the user. The user can then be presented with a treatment activity (e.g., at block 113 for the first skill or block 114 for the second skill, respectively) targeted to the identified disorder or impairment, and the user can again be tested after a treatment has been administered for an appropriate amount of time (e.g., one hour, periodically over a week or a month, etc.). If the user still cannot meet or pass the skill test (e.g., the first skill test or the second skill test), administration or presentation of the corresponding treatment may continue (e.g., at block 113 or block 114) until the user is able to pass the skill test (e.g., at block 212 the user tests within normal limits) or the treatment is otherwise halted by the program, a clinician or the user. In alternate implementations, the initial skill test can be utilized as the baseline value, and can proceed to the treatment directly thereafter (including testing after the skill test is performed and continuing the treatment until the skill test is met or otherwise stopped).

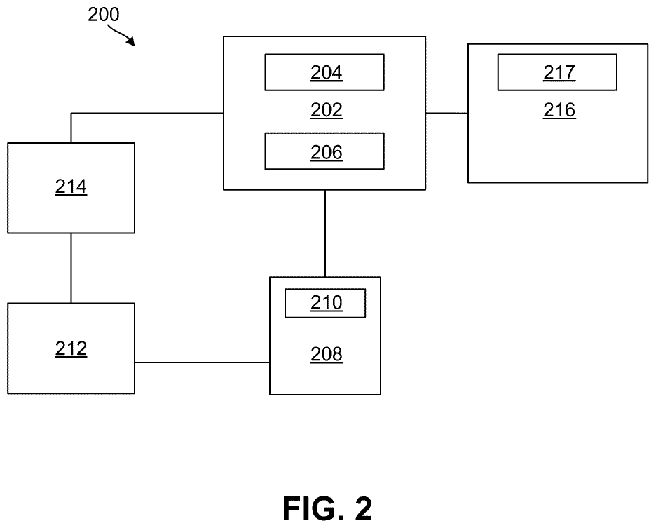

FIG. 2 illustrates a computer system 200 which can be configured to perform a process of diagnosing, assessing or treating a vision disorder afflicting a user 212, such as, for example, the process 100 of FIG. 1. The user 212 can be any person that can be a child or an adult. The system 200 includes a computing device 202 including at least one data processor 204 and computer-readable storage media (e.g., a memory) 206 coupled to the at least one data processor 204. The system 200 also includes a device 208 (e.g., a head-mountable virtual reality (VR) device) configured to communicate with the computing device 202 and having a display 210 configured to display images to a user 212 using (e.g., wearing) the device 208 such that the display is disposed in front of the user's eyes. As shown in FIG. 2, the system 200 can also include one or more input device 214 configured to acquire user input based on active input received from user 212 and/or based on passively acquired sensor image data. Non-limiting examples of the input device 214 include a mouse, keyboard, gesture/motion tracking device, microphone, camera(s), omnidirectional treadmill, game pad, body temperature monitor, pulse rate monitor, blood pressure monitor, respiratory rate monitor, electroencephalography device, or any other device. Information acquired by the one or more input devices 214 may be transmitted to the computing device 202, as shown in FIG. 2.

As also shown in FIG. 2, the computer system 200 can include or it can communicate via a remote connection with a server 216 which can include one or more databases 217 stored on one or more storage media and configured to store information acquired by the computing device 202 and other computing devices. The information, at least in part, can also be stored in the memory 206 of the computing device.

The computing device 202 can be any suitable computing device, such as a desktop or laptop personal computer, a personal digital assistant (PDA), a smart mobile phone, a server, or any other suitable computing device that can be operated by a user and can present services to a user. As mentioned above, the computing device 202 includes the at least one data processor 204 and the one or more computer-readable storage media 206. Computer-executable instructions implementing the techniques described herein can be encoded on one or more computer-readable storage media 206 to provide functionality to the storage media. These media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. It should be appreciated that, as used herein, a “computer-readable media,” including “computer-readable storage media,” refers to tangible storage media having at least one physical property that may be altered in some way during a process of recording data thereon. For example, a magnetization state of a portion of a physical structure of a computer-readable medium may be altered during a recording process.

The computing device 202 can be coupled to the device 208 via a wired or wireless connection. Similarly, the computing device 202 can be coupled to a controller (e.g., a touchscreen device coupled to the computing device 202) via a wired or wireless connection.

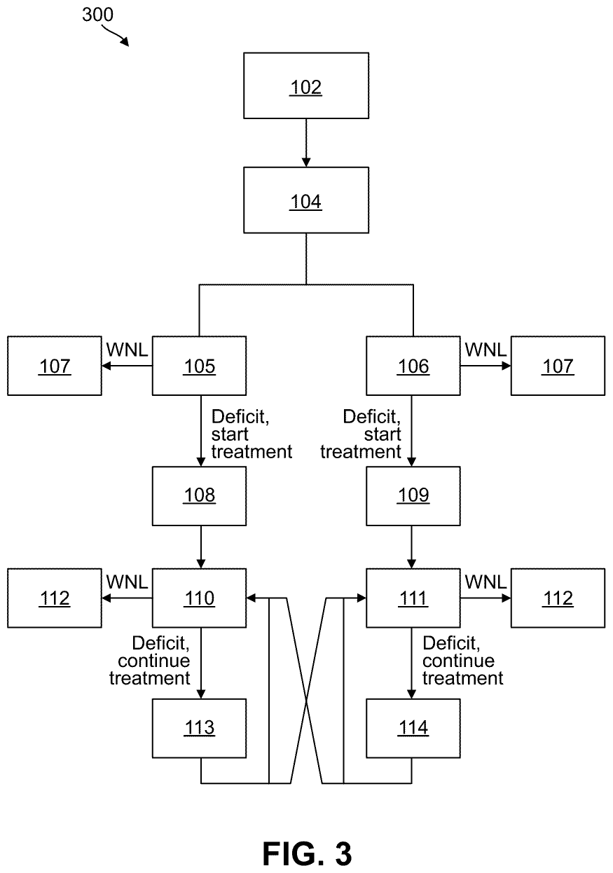

FIG. 3 illustrates a process 300 of operating a computing system to assess and/or treat a vision disorder of a person.

In general, the process 300 can be similar to the process 100 of FIG. 1. However, the process 300 depicts an interaction between the two tracks (e.g., block 105 and following blocks for the first skill, skill 1, or block 106 and following blocks for the second skill, skill 2), in which progress in one skill test is shown in FIG. 3 by new flow-chart lines added at the bottom of FIG. 1 to show that tests for Skill 2 (e.g., at block 111) may follow treatment activities for Skill 1 (e.g., at block 113), and vice-versa.

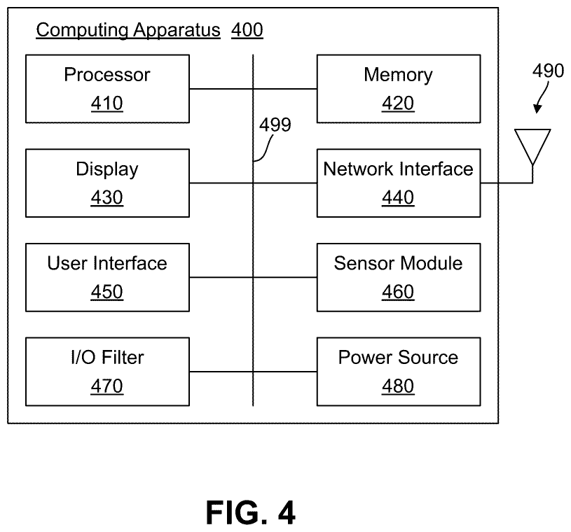

FIG. 4 illustrates an example computing apparatus 400 which may be used to implement one or more of the described features and/or components, in accordance with some example implementations. For example, at least a portion of the computing apparatus 400 may be used to implement at least a portion of the computing device 202, the server 216, and/or like. The components of the computing apparatus 400 can be implemented in addition to or alternatively from any of the components of the computing device 202 illustrated and/or described.

The computing apparatus 400 may perform one or more of the processes described herein. For example, the computing apparatus 400 may be used to execute an application providing for user control of a computing device in communication with the computing apparatus 400 and/or to provide an interface for the user to engage and interact with functions related to the computing device 202, in accordance with some example implementations.

As illustrated, the computing apparatus 400 may include one or more processors such as processor 410 to execute instructions that may implement operations consistent with those described herein. The computing apparatus 400 may include memory 420 to store executable instructions and/or information. Memory 420 may include solid-state memory, solid-state disk drives, magnetic disk drives, or any other information storage device. The computing apparatus 400 may include a network interface 440 to a wired network or a wireless network. In order to effectuate wireless communications, the network interface 440, for example, may utilize one or more antennas, such as antenna 490.

The computing apparatus 400 may include one or more user interfaces, such as user interface 450. The user interface 450 can include hardware or software interfaces, such as a keyboard, mouse, or other interface, some of which may include a touchscreen integrated with a display 430. The display 430 may be used to display information, such as information related to the functions of a computing device 202, provide prompts to a user, receive user input, and/or the like. In various implementations, the user interface 450 can include one or more peripheral devices and/or the user interface 450 may be configured to communicate with these peripheral devices.

In some aspects, the user interface 450 may include one or more sensors and/or may include an interface to one or more sensors, such as those described herein. The operation of these sensors may be controlled, at least in part, by a sensor module 460. The computing apparatus 400 may comprise an input and output filter 470, which can filter information received from the sensors or other user interfaces, received and/or transmitted via the network interface 440, and/or the like. For example, signals detected through the sensors can be passed through the filter 470 for proper signal conditioning, and the filtered data may then be passed to the sensor module 460 and/or processor 410 for validation and processing (e.g., before transmitting results or an indication via the network interface 440). The computing apparatus 400 may be powered through the use of one or more power sources, such as power source 480.

As illustrated, one or more of the components of the computing apparatus 400 may communicate and/or receive power through a system bus 499.

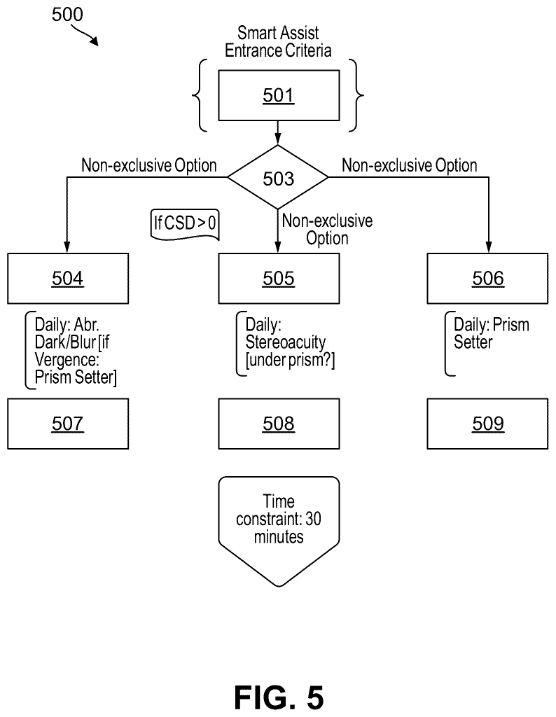

FIG. 5 depicts a flowchart for an example treatment process 500 for an example vision disorder. In some aspects, the process 500 (or at least a portion thereof) may be performed by one or more of the computing device 202, the server 216, the device 208, the computing apparatus 400, or the like.

The process 500 can start at block 501 where the apparatus 400, for example, may determine a user's vision condition, vision disorder, health condition, or the like. For example, the user 212 may undergo a full battery of tests, such as tests conducted by a physician at a clinic, tests conducted at a computing device, or the like. The full battery of tests may include a test set that evaluates one or more visual skills or capabilities that may be used to place the user into one or more treatment tracks. For example, a user may be given the following full battery test set: a prism tuner; a filter (e.g., filter test run with the prism from the prism tuner, if any); a stereoacuity test (e.g., with helping prism, without dark filter); a stereoacuity test (e.g., without the helping prism, without dark filter); a FourDot test; a vergence range test; a vergence facility test; or the like.

The process 500 may proceed to block 503 where the computing apparatus 400, for example, may determine a treatment track for the user 212. The determination may be based on the results of the full battery test conducted at block 501. As shown in the example of FIG. 5, the computing apparatus 400 may select from one or more different treatment tracks. For example and as further shown, the computing apparatus 400 may select from an Anti-suppression track 504, a Stereo track 505, and a Vergence track 506.

Depending on the treatment tracks selected, the computing apparatus 400 may determine certain activities (e.g., vision skills tests) for the user 212 to perform as part of the treatment track. As shown, the process 500 may proceed to block 507 as part of the Anti-suppression track 504. The block 507 may include anti-suppression entrance criteria which may define certain threshold skill levels to address the identified vision condition or disorder of the user 212. For example, to treat suppression, the computing apparatus 400 may prescribe binocular games and activities using a level of dark/blur filter that is assessed at the start of a session. The amount of filter may be adjusted based on the user 212's performance in the testing phase, and may be gradually reduced as the user demonstrates that it's no longer needed, which corresponds to moving through the stages of the track (e.g., track 504). The user 212 may be required to score above the threshold skill level during skill tests in order to move through the stages of the track 504.

As further shown, the process 500 may proceed to block 508 as part of the Stereo track 505. The block 508 may include stereo entrance criteria which may define certain threshold skill levels to address the identified vision condition or disorder of the user 212. For example, the stereo track 505 may include activities (e.g., skill tests) to improve or make sure the eyes are aligned, compensating for intraocular imbalance, and using stereo depth tests. The goal can be three-fold: improve the quality of depth judgments (by extracting depth from disparity signals when the eyes are aligned), promote the use of monocular neural inputs by the stereo-depth neural mechanisms that use those inputs, and give the brain reason to increase the weight that it gives to stereo relative to other depth cues (because stereo is in fact now trustworthy).

To treat lack of stereo, the sizes and disparities of the objects may be large at first, and may progress to smaller, and scaffolding may be used at first to help the visual system learn how to rely on stereo as the user demonstrates that it's no longer needed, which corresponds to moving through the stages of the track (e.g., track 505). The user 212 may be required to score above the threshold skill level during skill tests in order to move through the stages of the track 505.

As further shown, the process 500 may proceed to block 509 as part of the Vergence track 506. The block 509 may include vergence entrance criteria which may define certain threshold skill levels to address the identified vision condition or disorder of the user 212. For example, the vergence track 506 may include training at least four skills, including vergence facility (e.g., the ability to switch quickly to a new vergence eye posture), vergence range, tonic vergence (e.g., dark phoria), and vergence responses to accommodative stimuli. The computing apparatus 400 may measure tonic vergence (or dark phoria) but it is expected to move towards orthophoria on its own without explicit activities to target it specifically, as is frequently the case during treatment in clinical vision therapy settings. The computing apparatus 400 can optionally train appropriate vergence responses to accommodative stimuli. The activities may promote independent use of vergence and accommodation by holding accommodation fixed.

Clinically, one may utilize the base-in minus, base-out plus (BIMBOP) technique-which may be replicated in VR using inverted bifocal lenses (e.g., using the device 208).

To treat vergence, the range of vergence responses may be increased over time, interleaved with increases in the size of jumps the patient (e.g., the user 212) can make—with the absolute values of prism depending also on the patient's tonic vergence (dissociated phoria) as well as sustained alterations to vergence demand(s). Games and activities may include rapid changes to vergence demand, slow changes, or verge-and-hold changes. Thus, the user may improve by increasing vergence accuracy, range, and sustainability, respectively until the computing apparatus 400 determines the user has satisfied the appropriate threshold skill levels.

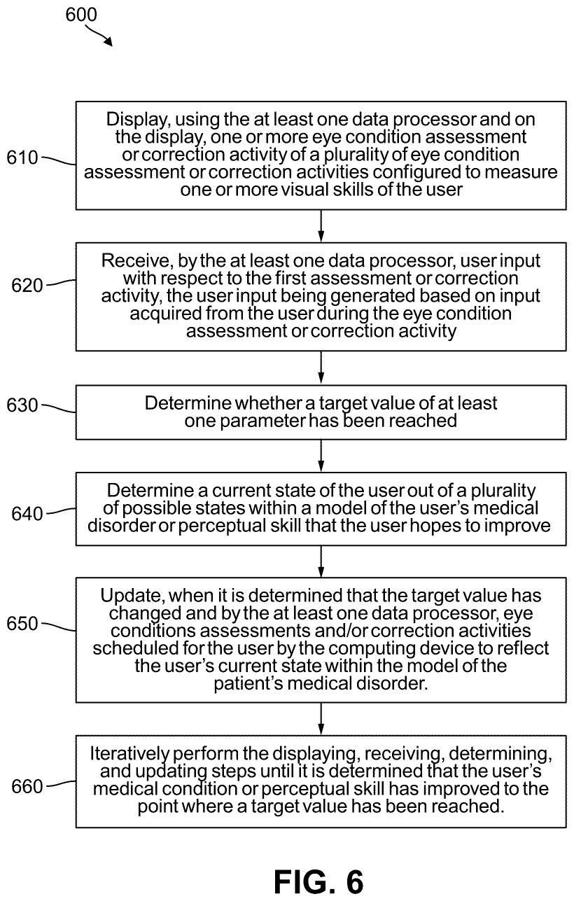

FIG. 6 illustrates a flowchart of a method 600 for vision assessment and correction therapy, in accordance with some example implementations. In various implementations, the method 600 (or at least a portion thereof) may be performed by one or more of the computing system 200, the computing device 202, the server 216, the computing apparatus 400, other related apparatuses, and/or some portion thereof. For example, the method 600 may be performed by a computer system for vision assessment and correction (e.g., the computing system 200). In some aspects, the computing device 202 includes the at least one data processor 204 and the one or more computer-readable storage media 206. The device 208 may be configured to communicate with the computing device 202 and may include the display 210. The display 210 may be configured to interact with the user having a medical disorder or vision perceptual skill. The vision perceptual skill may include a perceptual motor skill that the user hopes to improve. The at least one data processor 204 may be configured to execute computer executable instructions to perform the method 600. The medical disorder or vision perceptual skill may include Anisometropic Amblyopia, Strabismic Amblyopia, Intermittent exotropia, constant exotropia, Disorders of vergence, Convergence insufficiency, Convergence excess, Divergence insufficiency, Divergence excess, Esotropia—intermittent, Esotropia—caused by far-sightedness, Hypertropia, cortical suppression, lack of sensory binocularity needed for flat fusion, lack of sensory binocularity from vergence insufficiency, Accommodative insufficiency, Accommodative infacility, TBI, Anomalous correspondence, Oculomotor, Glaucoma (guidance for treatment, through testing), Low-vision training (eccentric viewing training, scotoma avoidance, specialty prism use [e.g. Pelli prism]), Brain Injury (including concussion, traumatic brain injury, or cerebrovascular accident), visual field loss, neglect, confusion, scotoma, lack of or impaired stereoscopic depth perception, Sports vision-related conditions (e.g., reaction time training, eye-hand or eye-body coordination), or the like.

Method 600 can start at operational block 610 where the apparatus 400, for example, can display, using the at least one data processor (e.g., at least one data processor 204) and on the display (e.g., display 210), a first eye condition assessment or eye condition correction activity of a plurality of eye condition assessments or eye condition correction activities configured to measure one or more visual skills of the user. For example, the apparatus 400 may perform or obtain results from a full battery of vision tests for a user (e.g., the user 212). The apparatus 400 may then display on a user interface (e.g., user interface 450 or the display 210) a first eye condition assessment or eye condition correction activity (e.g., a vision disorder, a vision condition, a vision correction activity, or the like) based on the results of the full battery of tests.

Method 600 can proceed to operational block 620 where the apparatus 400, for example, can receive, by the at least one data processor (e.g., at least one data processor 204), user input with respect to the first assessment or correction activity, the user input being generated based on input acquired from the user during the first eye condition assessment or eye condition correction activity. For example, the user input may include results from the full battery of tests that may be performed at block 501 or 102. The user input may also include results from the first eye condition assessment or eye condition correction activity (e.g., test performed at block 105, 106, 504, 505, or 506).

Method 600 can proceed to operational block 630 where the apparatus 400, for example, can determine, by the at least one data processor using the received user input, whether a target value of at least one parameter has been reached. The target value of the at least one parameter may be indicative of a perception of a first property of the first eye condition assessment or eye condition correction activity by at least one eye of the user. For example, the target value may include a threshold score or skill level achieved for a particular vision skill test (e.g., the first skill test or the second skill test at blocks 105 or 106).

Method 600 can proceed to operational block 640 where the apparatus 400, for example, can determine, based on the target value being reached, a current state of the user out of a plurality of possible states within a model of the user's medical disorder or perceptual skill that the user hopes to improve. For example, the computing device 202 may determine that the user 212 performed a vision skills test (e.g., the first skill test or the second skill test at blocks 105 or 106) within normal limits and no longer needs treatment. Examples of vision or perceptual skills that may be improved using the method 600 or the computing system 200 include: skills that are already in the normal range or above normal before treatment; vergence range; visual acuity (resolution acuity); visual acuity (dynamic acuity); contrast sensitivity; Central-peripheral awareness; stereo depth; visual processing; perceptual (visual concentration, visual closure, visual sequencing, visual motor integration, visual search, visual scan, visual span, or the like); reaction time; accommodation; tracking accuracy; flat fusion; stereopsis; awareness of diplopia; minimized fixation disparity; or the like.

Method 600 can proceed to operational block 650 where the apparatus 400, for example, can update, when it is determined that the target value has changed and by the at least one data processor, eye conditions assessments and/or correction activities scheduled for the user by the computing device (e.g., computing device 202) to reflect the user's current state within the model (e.g., treatment track) of the user's medical disorder. For example, the computing device 202 may adjust the target value (e.g., threshold skill level) for a particular vision skill (e.g., stereo depth). The eye condition assessments and/or eye condition correction activities scheduled for the user may include an eye condition assessment or eye condition correction activity from the plurality of eye condition assessments or eye condition correction activities configured to measure one or more visual skills of the user.

Method 600 can proceed to operational block 660 where the apparatus 400, for example, can iteratively perform the displaying, receiving, determining, and updating steps (e.g., blocks 610, 620, 630, 640, and/or 650) until it is determined (e.g., by the computing device 202) that the user's medical condition or perceptual skill has improved to the point where a target value has been reached. For example, the apparatus 400 may repeat skill activities and skill tests until the user 212 scores above a threshold level, where it is determined the user 212 vision condition or capabilities are within normal limits and no longer require treatment.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 1. The Smart Assistant Systems, Methods, and Devices

The purpose of this Example is to describe implementations of a vision assessment and correction system. In one aspect, a vision assessment and correction system such as the Smart Assist (SA) (including associated systems, methods, and/or devices) may be configured to provide an automated customized treatment for health disorders using a series of feedback loops where the end-user performs a test or series of tests that drive treatment activities.

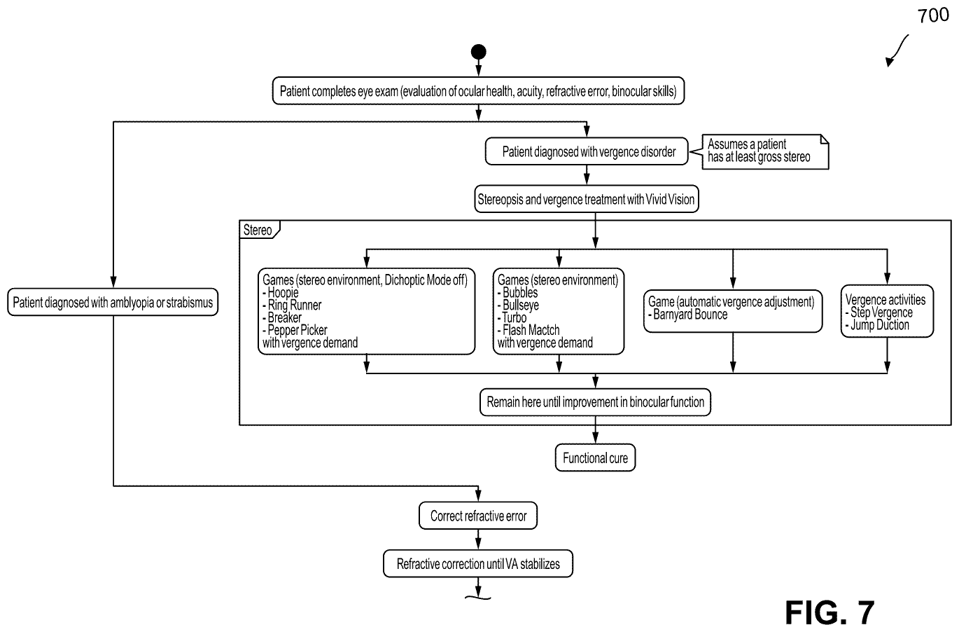

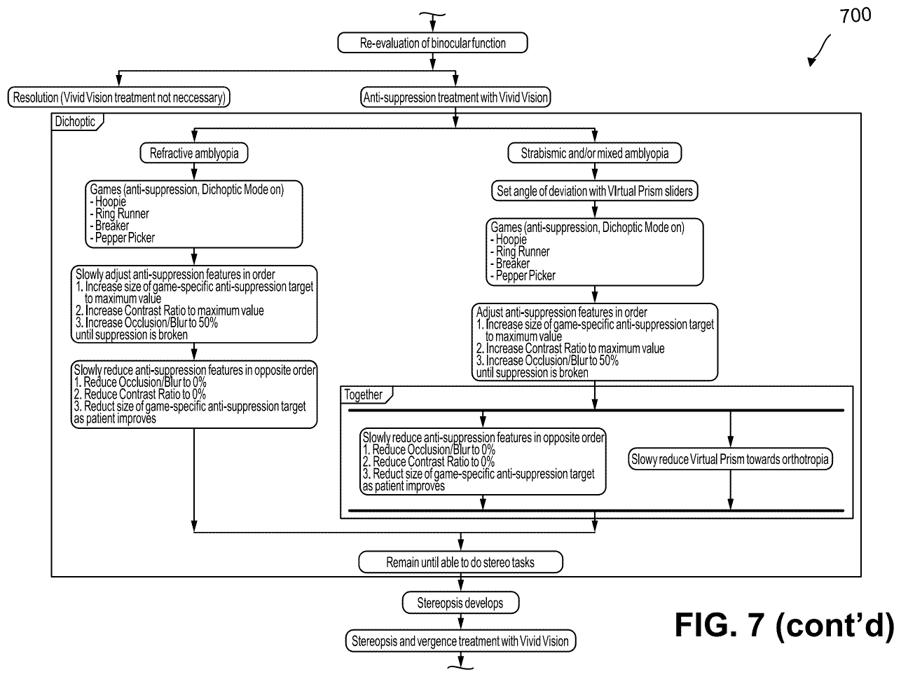

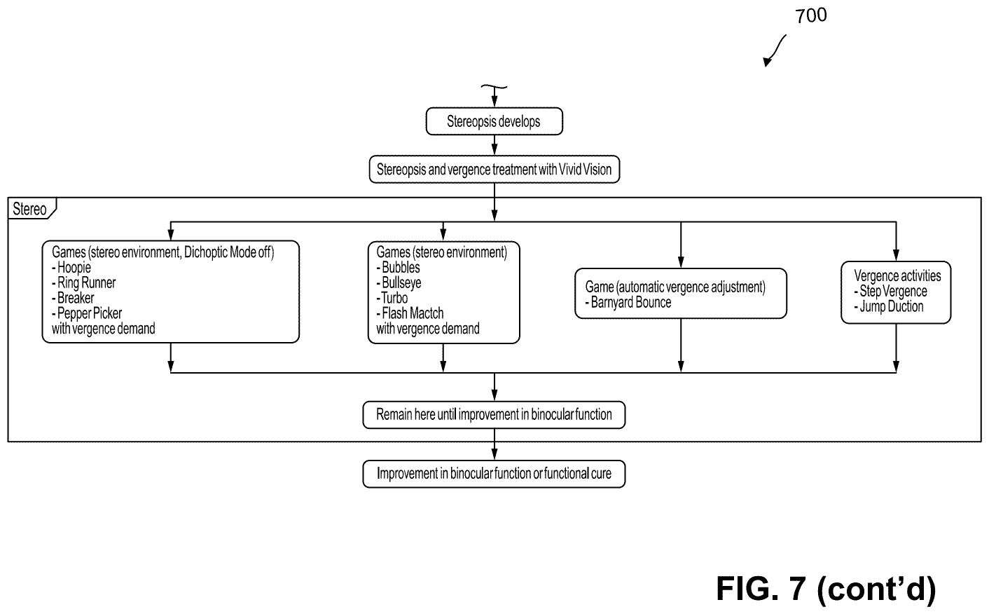

FIG. 7 shows an example flowchart of a treatment system consistent with implementations of the current subject matter. As shown in FIG. 7, an initial examination of visual function is performed. Next, patient diagnosis occurs, with the patient being diagnosed with amblyopia or strabismus, in which case the refractive error is correction until visual acuity (VA) stabilizes, or with vergence disorder. If a diagnosis of vergence disorder is determined, stereopsis and vergence treatment with VividVision is initiated. Stereopsis and vergence treatment is continued until there is an improvement in binocular function, leading to a functional cure for the vergence disorder. If, instead, refractive error is corrected until visual acuity stabilizes, there will be a second examination of visual function. As a result of this examination, there may be full resolution of visual function and no further treatment may be necessary. Alternatively, if the second examination does not indicate resolution of visual function, anti-suppression treatment with VividVision is initiated. Anti-suppression treatment is continued until the patient is able to complete stereo tasks. Once able to perform stereo tasks, stereopsis develops, and stereopsis and vergence treatment are initiated. As indicated previously, stereopsis and vergence treatment is continued until there is an improvement in binocular function, leading to a functional cure for the stereopsis (i.e. resolution of the binocular function disorder).

SA can include computerized systems and associated programming (such as e.g., an algorithm, or machine learning system) that guides a treatment plan. In exemplary embodiments, SA can be used to address binocular vision disorders, including but not limited to amblyopia, lack of stereo, strabismus, and/or vergence disorders. In some implementations, SA can also be used to treat accommodative disorders, or more generally, disorders of the visual system or body. Presently, the use of eye assessment and/or training systems can require vigilance and expertise on the part of the clinician, and as a result, treatments may not always be optimized for the patient. For example, clinicians may be unsure what settings to use or how settings should be adjusted during treatment and training activities when they use the product; similarly, a clinician may fail to adjust or alter treatment to continually challenge a patient's visual ability (or abilities), resulting in a treatment plateau. The SA system and associated systems, methods, devices, software, and/or algorithms described and illustrated herein can be beneficial as a guide for treatment whether it occurs in the clinic or at home. The home assessment and/or treatment application may include software to integrate the SA systems, software, and/or algorithms into an external device, for example the Launcher described herein, for real-time use of vision assessment and/or treatment it may be in not real-time, and can be integrated for clinical use or at-home guided treatment.

Patients will generally start utilizing the SA system after a clinic visit, in which the clinician prescribes use of the system. For example, the patient's first use can occur under supervision at the clinic, with mirroring of the display on the clinician's device (but without interactive Launcher use, in some implementations). This, however, is not required (but rather a suggested best-practice to observe the patient functioning in real time). In some embodiments, SA does not require this initial clinic visit. There may be some treatment decisions (i.e. programmed software choices/decision blocks, or machine-learning guided steps) that could be improved by incorporating the patient's diagnosis, which could allow the program to pre-select treatment pathways or activities known to benefit a specific diagnosis. In embodiments, which can be designed with Launcher and Home treatments in mind, patients can enter the same program regardless of diagnosis and the software can determine the training they need, according to their visual skills as measured by tests within the software.

An exemplary embodiment can be broken down into two stages: (1) addition of the vision tests that SA uses to assess patient status in order for the clinician to have better control over treatment, and (2) use of results from those tests to automatically control the treatment.

The patient's status can be assessed using tests within virtual reality (VR), augmented reality (AR), or mixed reality (MR), or on a computer with a suitable display such as a laptop computer, desktop computer, smart phone computer, or tablet computer, and the treatments can be provided to the patient according to their vision health or status. Treatment can be individualized by treating within specified Tracks. Exemplary Tracks can include: Anti-suppression, Stereo, and Vergence. Additionally or alternatively, other Tracks can include: Accommodation, Oculomotor, Random-dot stereogram perception, resistance to visual crowding, reading with the amblyopic eye, binocular reading, vertical vergence, cyclovergence, etc.

In embodiments, SA is designed to provide treatment that is safe, useful, and sensible even without active intervention from the clinician, but treatment can be under the clinician's control. To assist the clinician with this responsibility, the code creates reports for clinician review, raises flags to warn when a potential problem is detected (such as increase in tonic vergence away from ortho over time, or worsening of stereo depth perception or other deviation from expected values), and/or require clinician approvals in case of recommended actions in cases where SA would make a significant change in treatment (such as graduating the patient out of one of the treatment tracks).

Current vision testing and treatment systems often lack the ability to assess and train separate aspects/abilities of a user's vision system. Using the SA system and methods disclosed herein, individual aspects of a patient's vision (i.e., “tracks”) can be tested and/or trained separately or in a desired combination. The tests and/or training can be administered automatically (e.g., preset programs), or by clinical prescription and manual adjustments (e.g., programmed by a clinician or user, or a preset program adjusted by a clinician or user). In some examples, the programming is dynamic. For example, the programming may incorporate machine learning that enables tailoring of the system to the usage and/or visual ability of a specific user or patient over time (i.e., the programming adapts as the patient repeats use of the system/treatment program over time). Thus, the present system and methods disclosed herein enable a patient/user to receive vision assessment and training that is more likely to identify and improve the specific aspect of the user's visual system that is impaired, relative to conventional vision assessment and treatment procedures.

Treatment Approach

In embodiments, the SA approach to automated treatment includes Treatment Tracks and Testing.

Dysfunction in binocular vision can be caused by several different conditions, which compromises different sets of visual skills. Examples of vision conditions (disorders) are amblyopia and convergence insufficiency (CI). Examples of skills are regulation of interocular balance, use of binocular disparity for depth perception, and use of binocular disparity to control vergence and eye posture. Good binocular vision requires good function in all of these skills. To a large extent, treatment of the underlying conditions consists of improving function in skills that are deficient. The skills can to some extent be isolated and treated independently, which we do within separate Treatment Tracks.

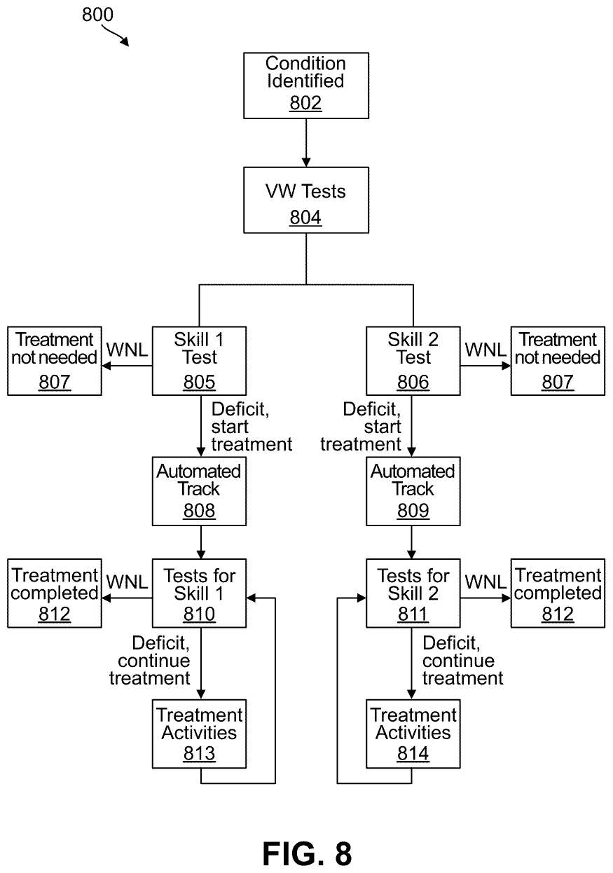

Treatment Tracks are illustrated in FIG. 8, and a more general treatment track overview is illustrated in FIG. 1 and described previously.

FIG. 8 is a process flow diagram 800 illustrating a method of operating a computing system to assess and/or treat a vision disorder of a person. The computing system can include at least one data processor and memory communicatively coupled to the at least data processor. The memory can be configured to store computer-executable instructions embodied as a vision correction platform or application. The computer-executable instructions, when executed by the at least one data processor, perform the described method. Furthermore, in some aspects, the entire or part of the platform can be stored on a remote computing device and can be accessible via the computing device. The computing system is in communication with a head-mountable display (e.g., a virtual reality (VR), augmented reality (AR), or the like), as discussed in more detail below.

As shown in FIG. 8, the process 800 can start at block 802, at any suitable time. At the block 802, a condition or an issue in a user or patient's vision can be identified by one or more tests of perception by the user, identification by a clinician administering a manual test, or automated identification by the user or the clinician. The process 800 may continue at block 804 where the computing system may assess the user or patient's vision. For example, the user may, at block 805, perform a first skill test or, at block 806, a second skill test to assess the user's vision. If the user can perform the skill (e.g., the first skill test or the second skill test) within normal limits (WNL), then at block 807, no further treatment may be necessary, and the system can proceed to testing another skill (e.g., the other of the first skill test or the second skill test, a third skill test, a skill N test, etc.). However, if the user cannot perform or pass the test (e.g., first skill test) to a sufficient level, then the system may proceed to block 808, and implement an automated track for treatment of a disorder or impairment identified/determined in the initial test (e.g., a disorder or impairment associated with a corresponding one of the first skill or the second skill at block 805 or block 806). If the user cannot perform the second skill test, at block 809, the system may implement an automated track for treatment of the disorder or impairment identified/determined in the initial test. In some implementations, the specified skill (e.g., the first skill test or the second skill test) can again be tested (e.g., at block 810 for the first skill test or block 811 for the second skill test, respectively) to generate a base line value for comparison to further (downstream) testing of the user. The user can then be presented with a treatment activity (e.g., at block 813 for the first skill or block 814 for the second skill, respectively) targeted to the identified disorder or impairment, and the user can again be tested after a treatment has been administered for an appropriate amount of time (e.g., one hour, periodically over a week or a month, etc.). If the user still cannot meet or pass the skill test (e.g., the first skill test or the second skill test), administration or presentation of the corresponding treatment may continue (e.g., at block 813 or block 814) until the user is able to pass the skill test (e.g., at block 812 the user tests within normal limits) or the treatment is otherwise halted by the program, a clinician or the user. In alternate implementations, the initial skill test can be utilized as the baseline value, and the process can proceed to the treatment directly thereafter (including testing after the skill test is performed and continuing the treatment until the skill test is met or otherwise stopped).

### Example 2. SA Treatment Method

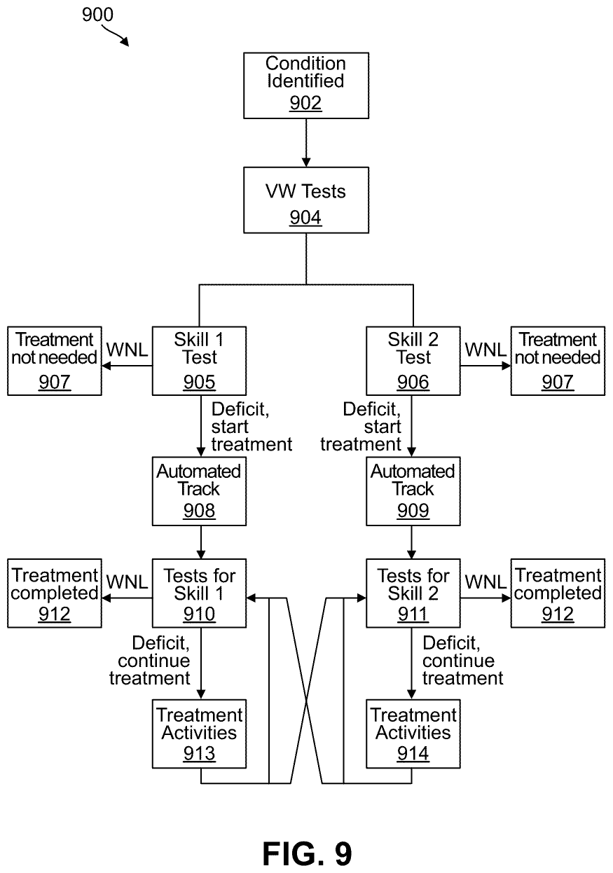

The purpose of this Example is to describe an exemplary embodiment of a generalized Smart Assistant (SA) method that can be carried out using the systems and methods disclosed and illustrated herein, and to provide a more detailed implementation of an example SA treatment method. An exemplary system for implementing the SA is illustrated in FIG. 9, and the computer system is more generally described in the discussion of FIG. 3 presented previously. First, a condition or an issue in a user or patient's vision can be identified by one or more tests of perception by the user, identification by a clinician administering a manual test, or automated identification by the user or the clinician. Next, the user or patient's vision is assessed via one or more treatment tracks. For example, the user can perform a skill 1 test or a skill 2 test. If the user can perform the skill within normal limits (WNL), then no further treatment is necessary, and the system can proceed to testing another skill (e.g., the other of the skill 1 test or the skill 2 test, a skill 3 test, a skill n test, etc.). However, if the user cannot perform or pass the test to a sufficient level, then the system will proceed to implementing an automated track for treatment of a disorder or impairment identified/determined in the initial test (e.g., a disorder or impairment associated with a corresponding one of the skill 1 or the skill 2). In implementations, the specified skill (e.g., skill 1 or skill 2) can again be tested to generate a base line value for comparison to further (downstream) testing of the user. The user can then be presented with a treatment activity targeted to the identified disorder or impairment, and the user can again be tested after a treatment has been administered for an appropriate amount of time (e.g., one hour, periodically over a week or a month, etc.). If the user still cannot meet or pass the skill test (skill 1 test or skill 2 test), administration or presentation of the corresponding treatment continues until the user is able to pass the skill test (or the treatment is otherwise halted by the program or a clinician or the user). In alternate implementations, the initial skill test can be utilized as the baseline value, and can proceed to the treatment directly thereafter (including testing after the skill test is performed and continuing the treatment until the skill test is met or otherwise stopped).

In embodiments, Treatment Tracks include Anti-Suppression, Vergence, and/or Stereopsis, and can additionally or alternatively include an Acuity track and an Accommodation track or others. It is reasonable to believe that by improving the skills that these tracks focus on, we will also see gains in other skills (acuity, accommodation) because the impediments to learning for these skills, caused by poor regulation of interocular balance, poor depth perception, and poor motor fusion, will be removed. For example, acuity in an amblyopic eye is expected to improve on its own over time, or with the help of activities outside of virtual reality (VR), due to the operation of routine learning mechanisms that maintain the neural mechanisms of vision, if retinal images fall on corresponding points in the two eyes (motor vergence) and habitual suppression of the weak eye is overcome (regulation of interocular balance). Accommodative range and facility are expected to improve if motor vergence improves, because there is direct input from the vergence system to the accommodative system, and because accommodation will then be the lone impediment to clear vision at near distance—at that point, improvements in accommodation cause improvements in vision, which was not the case before treating the motor vergence problem.

The ability to use one skill usually depends on another, which is why real-world experience is not sufficient to cure certain specific conditions. For example, seeing depth from binocular disparity requires that the eyes be properly converged, while planning an accurate binocular eye movement requires an appreciation of depth from disparity. The exact graph of dependencies between skills in a person with normal vision is not known, but from theoretical considerations and clinical experience it is clear that treating visual skills in isolation is beneficial. By analogy, if a person is not good at golf, they can improve their golf game by adding exercises that work specific muscle groups in isolation at the gym better than by simply playing more golf. The interaction between two tracks, in which progress in one is shown in the following figure by new flow-chart lines added at the bottom of the previous figure, to show that tests for Skill 2 may follow treatment activities for Skill 1, and vice-versa.

FIG. 9 illustrates a process 900 of operating a computing system to assess and/or treat a vision disorder of a person.

In general, the process 900 can be similar to the process 800 of FIG. 8. However, the process 900 depicts an interaction between the two tracks (e.g., block 805 and following blocks for the first skill, skill 1, or block 806 and following blocks for the second skill, skill 2), in which progress in one skill test is shown in FIG. 9 by new flow-chart lines added at the bottom of FIG. 8 to show that tests for Skill 2 (e.g., at block 811) may follow treatment activities for Skill 1 (e.g., at block 813), and vice-versa, in iterations until one or both treatment criteria are met.

Anti-Suppression Track

This track treats regulation of interocular balance. Good interocular balance is itself a combination of several different skills: appropriate regulation of binocular rivalry, equal contribution (over time, if not at all times) to fused images, and appropriate inhibition by a first eye of the second eye's inhibition of the first eye. These mechanisms may be correlated with each other, so that “relative strength” of one eye relative to the other is a meaningful concept.

The focus during treatment is to coax the brain into using information from the amblyopic eye, which we do by increasing the relative strength of input to the brain from the amblyopic eye through dark filter and blur of the fellow eye. Prism is used, if needed, to promote good binocular fusion of the stimuli during these games.

To treat suppression, the primary tool is binocular games and activities using a level of dark/blur filter that is assessed at the start of the session. The amount of filter is adjusted based on patient performance in the testing phase, and is gradually reduced as the patient demonstrates that it's no longer needed, which corresponds to moving through the stages of the track. Games to treat suppression can include displays that are in “dichoptic mode.” In dichoptic mode, a portion of the display is shown only to the amblyopic eye, and successful game play requires using these monocularly displayed element(s). The purpose of dichoptic mode is to encourage central vision in the amblyopic eye. The background is binocular, so the patient performs the treatment activity in a stereoscopic background environment. Dichoptic mode is used at the start of treatment for suppression, and stereo mode (to encourage central fusion and use of disparity visual) is used toward the end of treatment.

A “cue-scaffolding” or “training wheels” approach may be introduced in later stages of anti-suppression training or stereo training to encourage weighting of disparity cues as the patient begins to utilize stereoscopic vision. In this approach, binocular disparity is not the only cue available to the patient within the display to signify the depth relations needed to do the task. For example, a game such as Bullseye or Bubbles may require the use of stereo depth cues to select the closest object on each trial of the game. Additional depth cues such as motion parallax, interposition, relative size, etc. can be introduced to help teach the visual system to start using the correlated stereoscopic depth cue appropriately. The principle behind learning to use a perceptual cue, such as the use of the binocular disparity cue to construct perceived depth, is described in [Backus, B. T. (2011). Recruitment of new visual cues for perceptual appearance. Sensory cue integration, 101-119; Law, C. L., Backus, B., & Caziot, B. (2011). Improvement in Stereoacuity through Training with Correlated Cues. Journal of Vision, 11(11), 1016-1016; Godinez, A., Gonzilez, S., & Levi, D. (2020). Cue scaffolding to train stereo-anomalous observers to rely on disparity cues. Journal of Vision, 20(11), 300-300.] The disclosures of each of the foregoing references are incorporated by reference herein in their entireties.

Stereopsis Track

This track includes: making sure the eyes are aligned, compensating for interocular imbalance, and using stereo to do depth tasks. The goal can be three-fold: improve the quality of depth judgments (by extracting depth from disparity signals when the eyes are aligned), promote the use of monocular neural inputs by the stereo-depth neural mechanisms that use those inputs, and give the brain reason to increase the weight that it gives to stereo relative to other depth cues (because stereo is in fact now trustworthy).

To treat lack of stereo, the sizes and disparities of the objects are large at first, and progress to smaller, and scaffolding is used at first to help the visual system learn how to rely on stereo.

Vergence Track

This track can involve at least four skills, including vergence facility (the ability to switch quickly to a new vergence eye posture), vergence range, tonic vergence (dark phoria), and vergence responses to accommodative stimuli. We measure tonic vergence (or dark phoria) but it is expected to move towards orthophoria on its own without explicit activities to target it specifically, as is frequently the case during treatment in clinical vision therapy settings. We can optionally train appropriate vergence responses to accommodative stimuli. Our activities promote independent use of vergence and accommodation by holding accommodation fixed; clinically one would utilize the base-in minus, base-out plus (BIMBOP) technique-which we can replicate in VR using inverted bifocal lenses, such as those disclosed in U.S. Provisional Patent Application No. 62/990,335, which is incorporated by reference herein in its entirety.

To treat vergence, the range of vergence responses is increased over time, interleaved with increases in the size of jumps the patient can make—with the absolute values of prism depending also on the patient's tonic vergence (dissociated phoria) as well as sustained alterations to vergence demand(s). Games and activities may include rapid changes to vergence demand, slow changes, or verge-and-hold changes. Thus, the patient improves by increasing vergence accuracy, range, and sustainability, respectively.

“Correctives” provide relief to the visual system in case it cannot adequately exercise a skill. In the case of inadequate vergence ability, simulated base-in or base-out prism in VR can relieve a vergence demand that is not compatible with the patient's phoria. In the case of inadequate regulation of interocular balance, the image being shown to the suppressing eye can be filtered to make it darker, or of lower contrast, or blurrier. Anti-suppression activities are done using a dichoptic environment with relief from phoria (motor vergence demand), using virtual prism, as well as relief from suppression, using dark/blur filter in the suppressing eye (often referred to as the dominant eye). Stereopsis activities are also done with relief from both phoria and suppression. Vergence activities are done with relief from suppression (dark/blur in the suppressing eye).

At some point during training, tests in the SA system may show that the dark/blur filter is no longer needed, or that the added prism is no longer needed. At that point they stop being used by SA during training, to encourage further improvement under more natural conditions.

Skill Levels

For each skill, which is synonymous with a track, patient status can be quantified as a number (level), for example from 0 to 10 (10=normal). These numbers, one for each track, comprise the patient's visual skills profile. The skill level is determined by testing. The skill level determines which activities occur during a session within that track. The underlying algorithm for computing a skill level is different for each skill, but all are normalized from 0 to 10 to make it easier for doctors and patients to understand. Normalizing the score may involve scaling test scores, and combining results from different subcomponent tests.

Use of level to determine the activities and settings in a session. For the Anti-suppression and Stereopsis tracks, the choice of games and activities, and the settings within the games and activities, can be determined by the skill level (together with the amount of dark/blur and prism, if needed). For the Vergence track, the choices and settings can be determined by the level, the tonic vergence and prism-required at 200 cm as assessed by the Prism Setter test.

Testing

Testing can occur for two reasons: (1) to control the treatment so as to make it appropriate during training and/or (2) to assess patient progress. To control the treatment, two tests are done at 200 cm at the start of each session: Prism Setter, and Dark/Blur. In embodiments, we can do both tests at the start of every session for all patients, even if the tests have consistently shown no need for one or the other type of relief (later we can stop a test if not needed). Alternatively, one test can be selectively excluded.

To assess patient progress and set a baseline, a series of tests may be performed at the start of treatment, which may last for weeks or months, and again at scheduled intervals during treatment. These scheduled tests may be assessment sessions, which are designed to be performed to completion in one setting. The output of the initial assessment would guide initial treatment activities. The output of the assessment sessions would guide ongoing treatment activities. The followings tests are proposed (i.e. the “full [test] battery”) for embodiments of the presently described systems and methods:

The Prism Tuner test may include non-fused phoric/tropic posture estimate followed by a fused trial series to estimate the patient's need for virtual prism. The output is a prism diopter value (i.e. horizontal and vertical prism) and degree value for rotation (in embodiments, the system may also display the degree conversion for horizontal and vertical prism, however many clinicians prefer to see the prism diopter value).

The Anti-suppression Filters test may include a dichoptic test to estimate the needed dark and blur filter combination to help a patient break suppression and succeed in the dichoptic games. The output is a value of blur filter and dark filter (scaled 0-100 for each), which then becomes the combined filter.

The Stereoacuity test estimates a patient's stereoacuity threshold; the test is designed to be performed without virtual prism as well as with the virtual prism values determined by Prism Tuner or manually entered by the clinician. The output is an arc second values as well as composite stereoacuity score (CSD) score scaled in whole numbers from 0 to 30.

The Four Dot test includes an estimate about suppression status. The output is a metric of suppression, fusion, or diplopia as well as the orientation of the diplopia (if applicable).

The Vergence Ranges test estimates the patient's maximum vergence value using either flat or stereoscopic stimuli. The output is a value in prism diopters.

The Vergence Facility test estimates the patient's ability to alter vergence in response to a change in vergence demand. The output is a value in completed cycles per time unit (30 or 60 seconds) at the tested demand (e.g. 15 cycles in 60 seconds at 3Δ BI/12Δ BO [where Δ indicates the prism diopter value]).

To control the treatment, the skill level can also be determined. In the Anti-suppression track, the Skill Level is determined by dark/blur filter (levels 0-8), so no additional tests are necessary. At levels 9 and 10, the stereopsis test is also needed, so Stereoacuity is run after Prism Setter and Dark/Blur if the Anti-suppression track is on and the skill level was determined to be 8 or above during Dark/Blur. In the Stereopsis track, the skill level is determined by the Stereoacuity test, which runs at the start of every session for that track (either with or without prismatic compensation, as determined by the Prism Tuner test). In the Vergence track, the skill level is determined by the Vergence Range and Vergence Facility tests (both). In this track, advancing to a new skill level depends on having both sub-skills (range and facility) at the required level. Only one of these skills changes at a given increase in level, so in principle only one of the tests is necessary, but for First Embodiment we will collect data for both at the start of each session. Note that the endpoints (vergence demands in prism diopters) of the Vergence Range test for a given level depend on the patient's tonic vergence and prism settings (as determined by Prism Setter). The endpoints used during the Vergence Facility test at a given level are significantly closer together than the range that was measured using the Vergence Range test in order to be sure the test is comfortable; the values for this test are determined by tonic vergence and prism setting from Prism Setter, but also from the history of previous Vergence Facility tests.

In particular, the Vergence Facility test is switches between endpoints, and the Vergence Range test roves between them, where the endpoints include the prism setting as one endpoint and extend in the direction of the tonic vergence posture, until the prism setting is 0, at which point it includes both BI and BO in a ratio of approximately 1:4 until it has been extended to 5 BI and 25 BO.

Note that in embodiments, patients cannot start the Vergence Track until their CSD Score is 10 or greater, because these patients won't be able to perform the Step Vergence and Jump Duction activities (these activities have not yet been adapted to allow responses based on flat-fusion, but could be adapted to do so in certain embodiments).

In addition to determining how much dark/blur and prism relief to use, the Dark/Blur test determines if the Skill Level in the Anti-suppression track (for levels 0-8) is sufficient to update the skill level, and the skill level is used to control treatment. The three skill levels comprise the patient's skill profile. Thus, these daily tests are sufficient to provide a snapshot of the patient's ability.

### Example 3. Clinical Alert

The purpose of this Example is to describe an implementation which includes a separate product or additional feature of the system can check whether a clinician who does not use SA, but instead uses a Vivid Vision (VV) system in manual-control mode, is prescribing treatments that are sensible, and alert the clinician that something may be abnormal or unusual in the way they are using the product. There is overlap in the automation of expert judgment needed to monitor patient performance during use of SA and clinician performance. In addition, the clinician produces data in the form of their prescription of activities. These prescriptions can be monitored for quality and conformity with best practices. The use of VV by the clinician also provides a source of information about how experts use VV to treat specific visual conditions, and this information constitutes expert knowledge that can be incorporated into SA.

Interface

Clinical Launcher

In embodiments, SA in the clinic will appear as a slightly different product than its incorporation into VV. Ideally, the program will offer a calibration step for the tests (e.g. anti-suppression, vergence demand, virtual prism, etc.) to the user (clinician/therapist), rather than focusing on the different treatment tracks. In other embodiments, the clinical SA can also focus on treatment tracks. In both embodiments, the SA should be intuitive and easy to use/enable (readily available option/no need to search for the option to use SA), as well as simple to disable.

A Risk Statement will be shown prior to turning on SA for a patient, to educate the patient of risks associated with SA treatment.

In embodiments, the SA system and associated programming and methods are designed to assist in the care of patients with amblyopia, strabismus, and vergence disorders (the Vivid Vision product has an indicated use as a haploscope). SA assesses a patient's status by using a series of tests within virtual reality (VR). Treatment is individualized based on a patient's test results, and can be updated periodically, such as every day or periodically at regular intervals. The treating clinician can be responsible for reviewing progress and adjusting, altering, or stopping treatment as necessary. Exemplary precautionary statements can be included in a user manual.

When performing a clinical session, at least the following five different scenarios may occur. Scenario 1: Patient inactive in Home and no test/SA data; clinic wants to run a completely manual session without running any of our tests first. Action: clinic runs manual session (no tests used, software used as traditionally used). Scenario 2: Patient inactive in Home and no test/SA data; clinic wants to use tests/SA to guide treatment during the clinical session. Action: Click on a button to run some or all of the battery tests. SA will do a battery of tests, then pop up with the activities the patient will do. Clinicians can uncheck items they don't want the patient to perform. Result: patient now has status within each track, settings for prism and dark/blur (which can now be modified), suggested parameter settings for games, and some or all test results. In the event that a clinician wishes to manually adjust activity or game features, they may have access to a full list of features or to a reduced list of features, or else features for the activity or game may not be available to them for adjustment at all. Scenario 3: Patient active in Home w/Smart Assist track data; clinic wants to run a completely manual session during the clinical session. Action: clinic runs manual session (no tests used, software used as traditionally used) and the patient's home treatment program follows the SA algorithm. Scenario 4: Patient active in Home w/Smart Assist track data; clinic wants to continue SA (pick up where patient is on the current SA tracks) during the clinical session. Action: patient logs data in the clinic, which functions as a standard SA session (i.e. the session in the clinic is identical to the next planned SA session in the patient's treatment). Scenario 5: Patient active in Home and Smart Assist; clinic wants to import prism and dark/blur settings that are consistent with SA and continue manually. Action: import the most recent settings from any SA test.

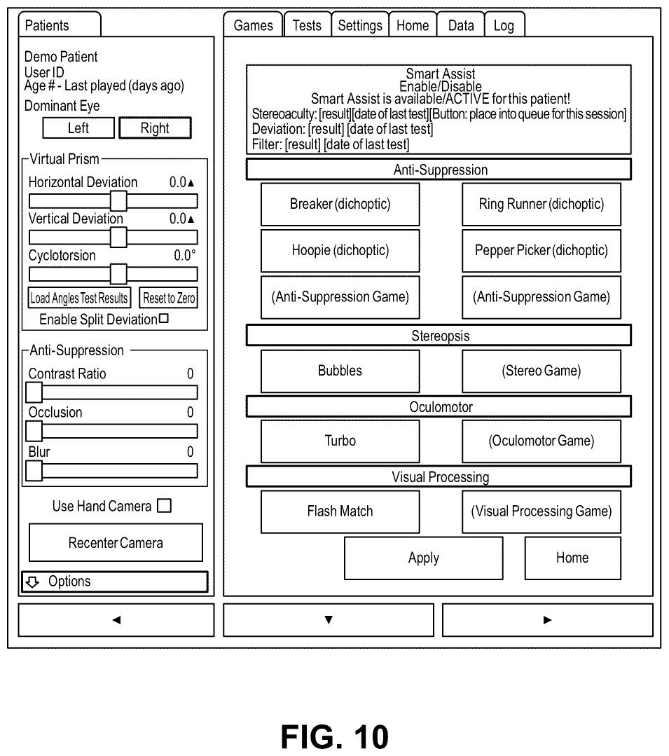

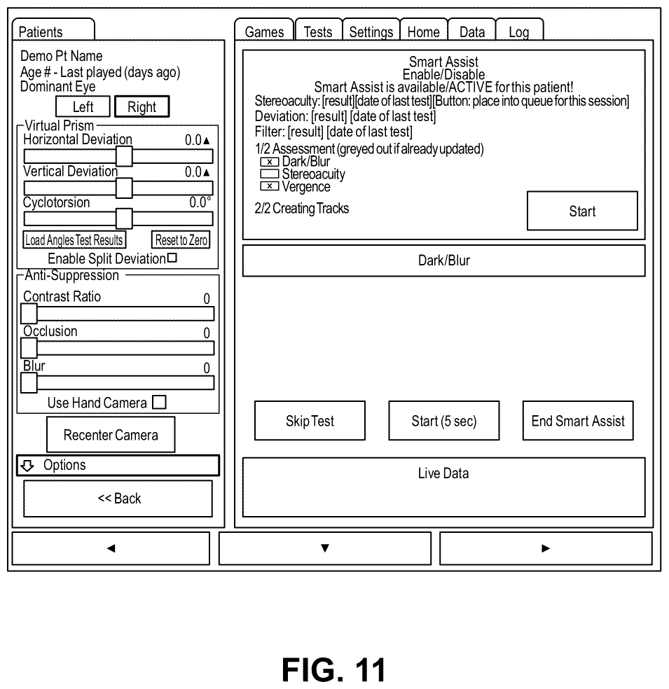

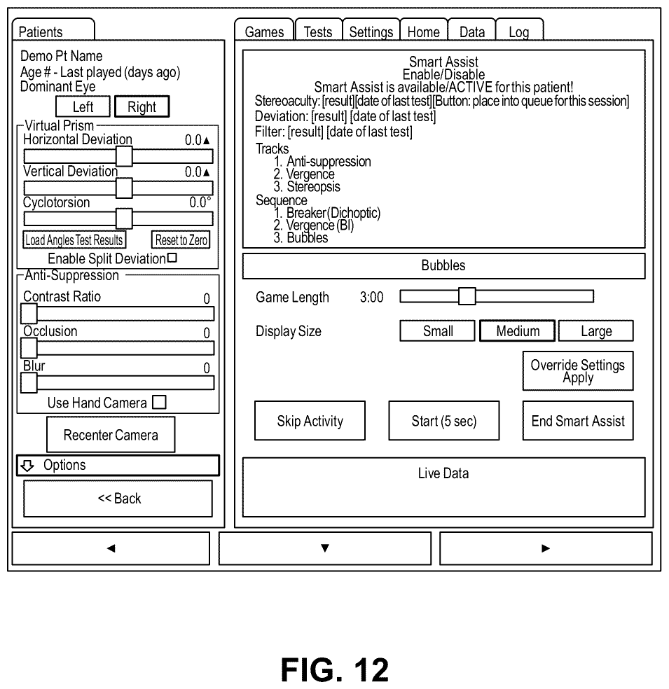

FIGS. 10-15 described below illustrate an exemplary Template for an SA Interface. FIG. 10 shows a user interface screen with all games available to a patient. FIG. 11 shows a user interface screen after a patient or user has selected the anti-suppression track/games, specifically the dark/blur test. FIG. 12 shows a user interface screen after a patient or user has selected the stereopsis track/games, specifically the bubbles game.

For example, Smart Assist can suggest a Session Plan (set of programmed activities) within the Session Maker, for the VV session.

Clinicians can either stay in the Session Maker or else run individual activities. If they stay in the Session Maker, the clinician can modify the Session Plan before it starts by adding and removing activities, and changing settings within the activities. The default Session Plan is the same as using Smart Assist at home.

If the session is being run from a Session Plan, then during each activity there is a pop-up that shows time elapsed and time remaining, and allows the clinician to skip the rest of the activity or modify the filters/prism settings on the fly. If skipped, you have two options: Start the Next Activity, or Exit Session Plan. When an activity ends naturally, use a countdown menu to give the clinician time to press Start Activity Now, Skip Activity, or Exit Session Plan. The Session Plan is shown as a list with check boxes that are checked as items are completed (or replaced with X if skipped, or a pie chart icon if only a percentage of planned time was completed).

Data Review and Monitoring Treatment Progress

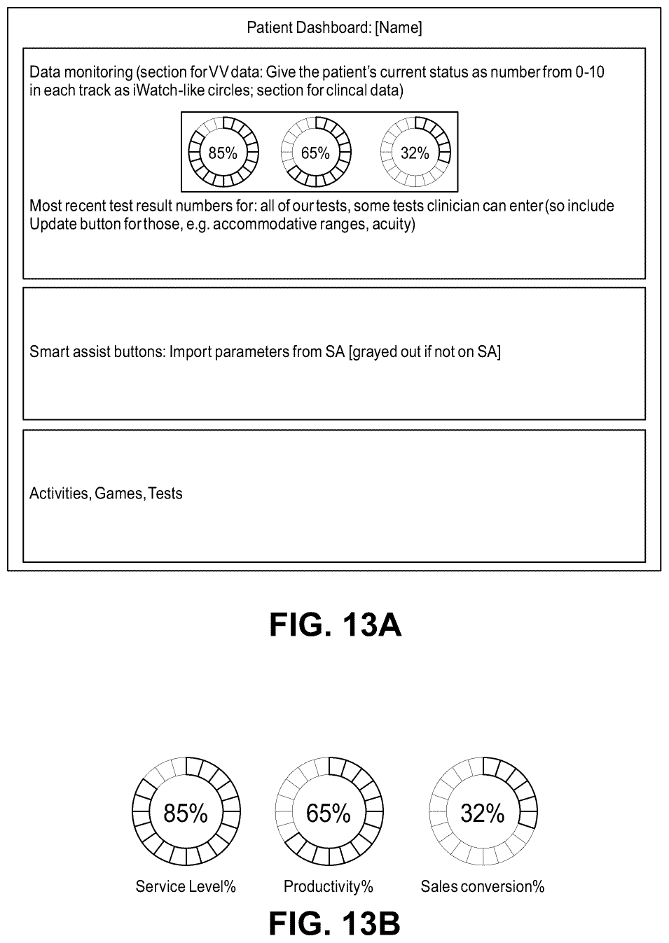

An interface for data review and monitoring of treatment progress may be incorporated into the SA system. This interface may include a progress bar or progress wheel or comparable interface display element that shows how a patient is progressing through the treatment steps. This may visible to the clinician, the patient, or both. FIG. 13A shows an exemplary user interface for data review and monitoring of treatment process. FIG. 13B shows an exemplary progress wheel for several test parameters.

Examples of a progress wheel may display days played (i.e. the wheel may be segmented and fill up pieces each day to get to test day), may show progress in SA track (Suppression, Stereo, Vergence), may show percent complete overall, or may show a percentile rank for a specific activity.

Patient Portal

The patient may be able to view data collected by the SA system and/or associated programing using a “patient portal”. The patient portal may display data in the same format that it is displayed to the clinician or it may be simplified or otherwise altered to aid the patient user in the interpretation of results. This data may be available in the human mortality database (HMD) or via a dedicated patient web portal to be viewed on a separate computer display device. The patient may be notified of results on a predetermined basis (i.e. daily, weekly, every n-th session, etc.)

Information/Reports Available to the Patient

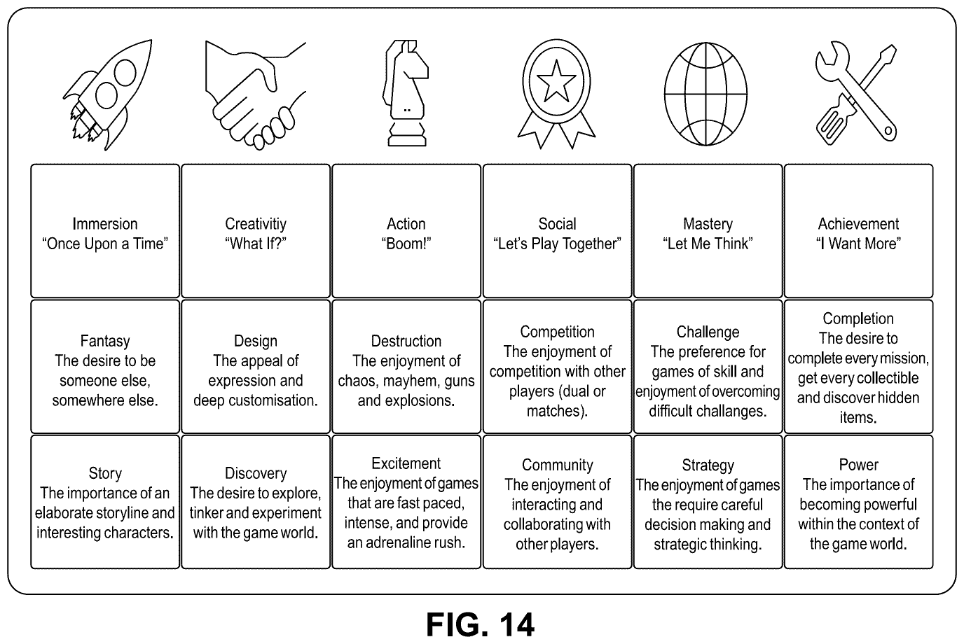

A Goal of the system may be Motivating the patient to complete tests or games. FIG. 14 shows an exemplary motivational scheme involving six different areas of possible motivation, and two satisfactory outcomes of each area of possible motivation.

In addition to showing the patient their data (mastery/achievement and/or areas that need improvement), SA takes advantage of a number of concepts traditionally applied in game design. Badges can be awarded based on both time invested and results achieved. Comparisons to other players, by showing percentile rankings, can provide competition. The game mechanics present in the VR environments provide fantasy, story, discovery, and action. By motivating the player during each activity within each session, and also in between sessions we help guide the patient through the entire automated treatment journey.

Motivation can also occur through changes in difficulty level of each task. The patient may take an easy-to-hard approach so that task is challenging, but doable. Additional aspects of treatment are described herein.

### Example 4. Separation of Visual Difficulty and Game-Play Difficulty

The purpose of this Example is to describe implementations of game use in a Smart Assistant (SA) system. In embodiments, SA can user games and activities that are designed to exercise visual skills while being engaging to play. Control over the difficulty of the game is one aspect of engagement. For many games, it is possible to make an operational distinction between “visual difficulty” and “game play difficulty”. By segregating these two types of difficulty, they can be controlled separately. For example, the Breaker game can be used to treat suppression (improper regulation of interocular balance). In the Breaker game, the user hits a virtually rendered ball using a virtually-rendered paddle. In dichoptic mode, the ball is shown monocularly to the non-dominant eye. A person being treated for suppression will attempt to hit the ball when it is small in size, but will not be able to do so if it is too small because they cannot see it. Therefore, an example of controlling the visual difficulty is controlling the size of the ball's image. When the ball's image is made smaller, the visual difficulty is increased, because the user's visual system is more likely to suppress the image of the ball. An example of controlling game-play difficulty is controlling the speed of the ball. As the speed is increased, the game becomes more difficult to play for any player, not just a player with improper regulation of interocular balance. This segregation of difficulty into visual-skill and game-skill allows the program to be used appropriately by users of many different visual-skill levels and game-play skill levels. The user's age is a factor in game-play skill level, so segregation allows the same game to be played by patients of different ages. Additionally, the patient may be given control over their own treatment plan, even when visual-skill difficulty is under computer control, by allowing them to adjust their own game-skill difficulty. This type of control by the patient can be an intrinsic motivation for the patient by adding an additional challenge with a goal to improve from session to session and over the entire course of treatment.

Treatment Using SA in the Clinic (VV Clinical Integration)

The SA system and/or program may be used in the clinic. By running the test battery, the clinician or therapist is afforded baseline data to optimize treatment for the patient in-office. Additionally, this testing data may be tracked over time to show improvement in a patient's visual skillset. The program may suggest settings for a specific game or activity that may be imported (based on test output) or overridden by the clinician/therapist. The option to override the suggested settings is an imperative component of the therapeutic process. Often in a therapy session the clinician or therapist alters an activity or an activity setting to intentionally challenge a patient. The dichotomy of suggested settings along with the ability to override settings manually affords the clinician a level of expert control over the patient's treatment.

Treatment Using SA at Home (VV Home Integration)

Perceptual learning is a component to the visual rehabilitation process. The suggestion is 5 times per week as the recommended number of non-office therapy sessions. The SA program can utilize abbreviated test sessions to “fine tune” game settings for a daily therapy session and full testing session every n-th session. This testing session becomes a tracking metric to review a patient's progress during home-based treatment.

The program provides a patient with options for game selection, affording the patient a level of inclusiveness in the therapy plan. The patient may also select certain game settings that affect the videogame difficulty of the task. The key component is that the videogame difficulty is segregated from the visual skill treated during the task. A patient should have visibility into time spent using the program-which acts as an additional motivation component. The ability to unlock badges or a like reward system for active participation/completion of therapy sessions may further motivate the patient to complete treatment sessions.

A limited data set that is easily understandable to the lay user is a component of the SA program. A more technical data set should be available to the clinician.

Maintenance Mode

After the patient has been treated, SA can be put into maintenance mode. As compared to the treatment phase, when SA is in maintenance mode, treatment time is reduced (for example, it could be reduced from five sessions per week to one session per week). The patient can also re-engage their use of SA when they feel it would be beneficial to do so. Billing for SA may be different during treatment and during maintenance, for example unlimited use during treatment, but for maintenance mode, a maximum of 10 hours/month (or other predetermined number).

For rests during treatment with SA, SA can allow unlimited use with a notice (suggestion) to “take a break” if the amount of uninterrupted gameplay and testing time exceeds a predetermined amount of time, for example 20 minutes.

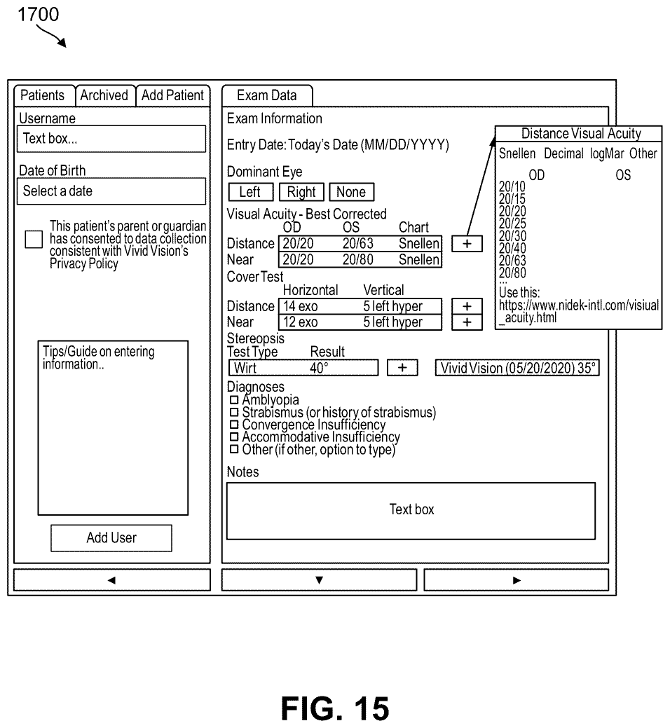

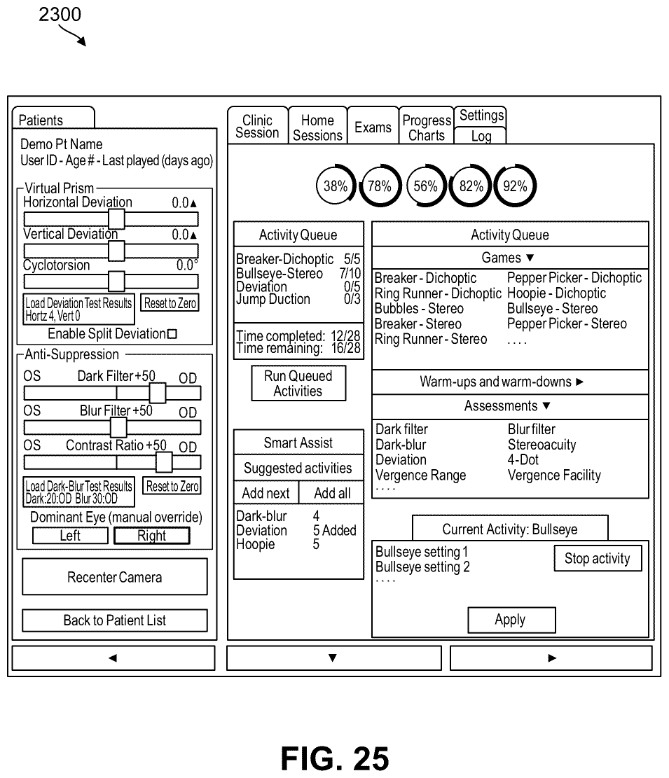

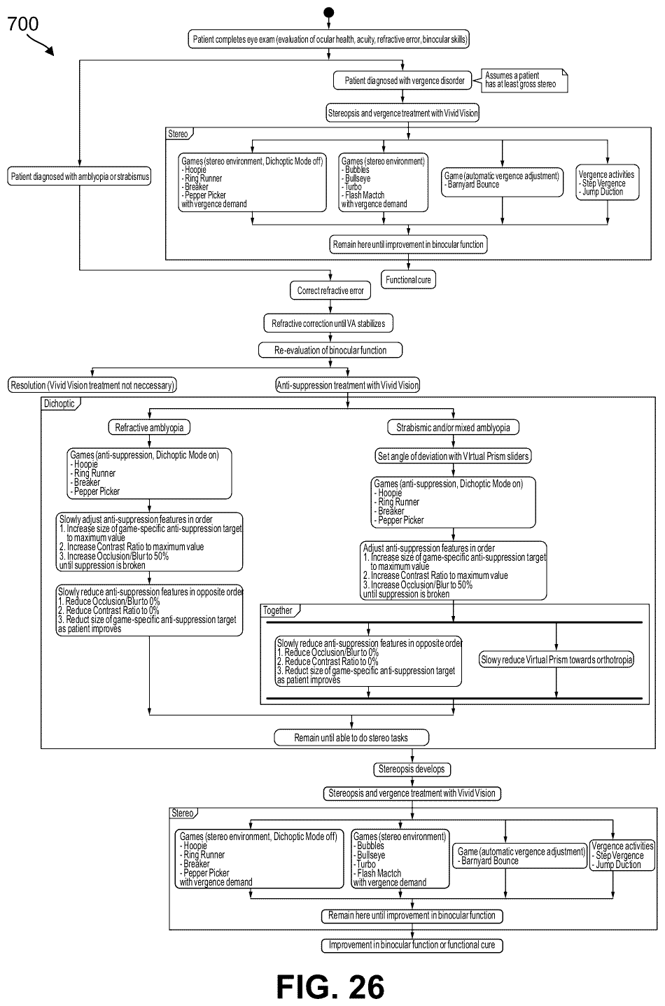

Storyboards for an exemplary user interface are provided in FIGS. 15-25.

FIG. 15 illustrates Adding a patient via a user interface.

When navigating to the Add Patient option, the tabs disappear and an option to input user (patient information) appears on the left side. The tabs are replaced with a new page that allows the clinician (VT) to input additional information related to the patient.

This information would be editable, but not within the Add Patient. The page is next available (upon Adding the User) AFTER the patient is selected from the Patients Tab, by selecting the Play Tab available along with other tabs such as Games/Activities/etc.

Exemplary items for the left side of the user interface screen include: Username, date of birth (DOB), GDPR checkbox (dynamic text based on DOB), General user instructions/tips, and an Add User button.

Exemplary items for the left side of the user interface screen include: Date (current date in MM/DD/YYYY or DD/MM/YYYY depending on region), Dominant Eye (change to: Left, Right, or None (if None, this gets set to Right in the software to capture patients with no Dominant Eye, and no filter can be applied)), Visual Acuity as measured by a chart (charts may include Listbox, Snellen (20 ft), Metric (6 m), Metric (4 m), Decimal, LogMAR/ETDRS, Landolt C, Tumbling E, or Other (for example this option may bring up space to enter name, and text box to enter value) or as measured by OD/OS values. Values offered in the OD/OS options should follow: website nidek-intl.com/visual_acuity (e.g. if Chart=Snellen (20 ft) then OD and OS will have options for 20/10 to 20/400, >20/400 as upper bound). Additional items for the right side may include Cover Test, Stereopsis (Near), Diagnoses (for example, amblyopia, strabismus (or strabismus history), convergence insufficiency, divergence excess, accommodative disorder, etc.), and a data entry page (e.g. at Baseline, then once a month add notes such as “patient results were last updated on DATE (e.g. MM/DD/YY).” Additionally, the interface may provide spaces to enter new data as well as a “Later” button. For example, a “Later” button may be provided, for optional use by a clinician, to prompt the system to ask the clinician for data to enter into the spaces for new data at a later date, such as the next time the patient is using the software. For stereopsis, the interface may specify the type of test (e.g. random dot, circles, fly, lang, frisby, ASTEROID, etc.), and a result (e.g. 12.5, 15, 20, 25, 30, 40, 50, 60, 80, 100, 150, 200, 400, 600, >600, etc.).

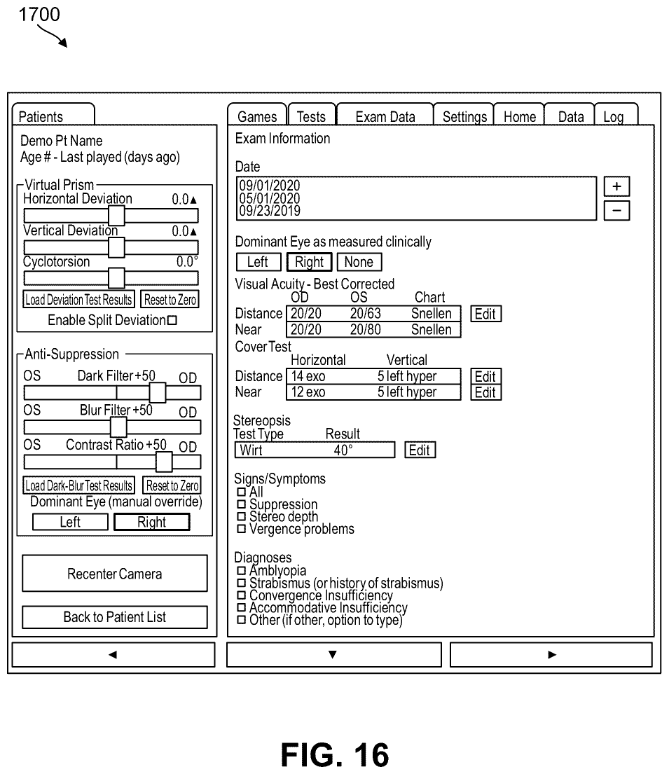

FIG. 16 shows an example of an updated left side of the Launcher interface, to include Smart Assist notes.

Note the following changes in the interface of FIG. 16 versus that of FIG. 15: Smart Assist notes under User ID include VV Test (by last full test date), last update to Exam Data page; Dominant eye changed to Dominant Eye (Manual Override) and moved into Anti-Suppression field; Filters re-ordered—Dark Filter/Blur Filter/Contrast Ratio; Filters changed to mimic Virtual Prism sliders—center 0, −100 left, +100 right; −100=most dense filter OS, +100=most dense filter OD; +50 would be a +50% filter OD, etc. The slider should include an OS/OD indicator as noted in the drawing; Option to Load Dark-Blur test results and Reset (filters) to Zero (again, similar to virtual prism).

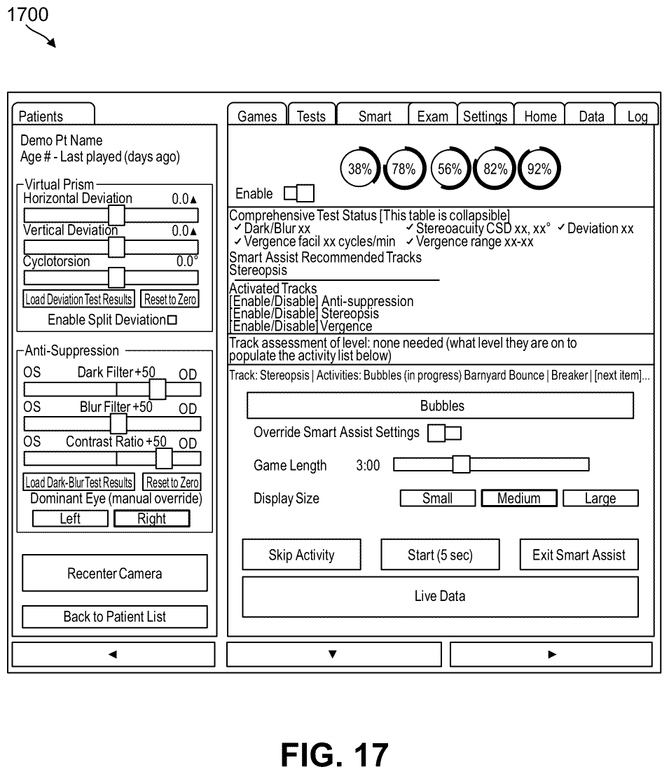

The Smart Assist functionality may be implemented alongside manual operation controls, for example it may be represented by a separate tab within the software. Alternatively, the Smart Assist items may be combined with the manual controls included in the current Games and Activities tabs into one single tab and categorized as is the current layout. An exemplary interface 1700 is shown in FIG. 17. The interface 1700 may include, in some embodiments, a Smart tab 1705, a Manual tab 1710, a Clinic or Session Builder tab 1715, and a Progress screen 1701 to show treatment tracks and current completion status. Additionally or alternatively, the interface 1700 may be configured to include a Toggle feature to enable and disable Smart Assist.

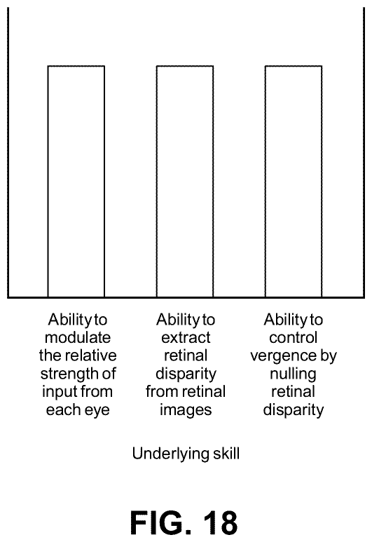

FIG. 18 shows a graph of three exemplary underlying visual skills. The underlying skills are, from left to right, ability to modulate the relative strength of input from each eye, ability to extract retinal disparity from retinal images, and ability to control vergence by nulling retinal disparity. These three underlying skills may be tested by the tests and games described herein, and using the interfaces and systems described herein.

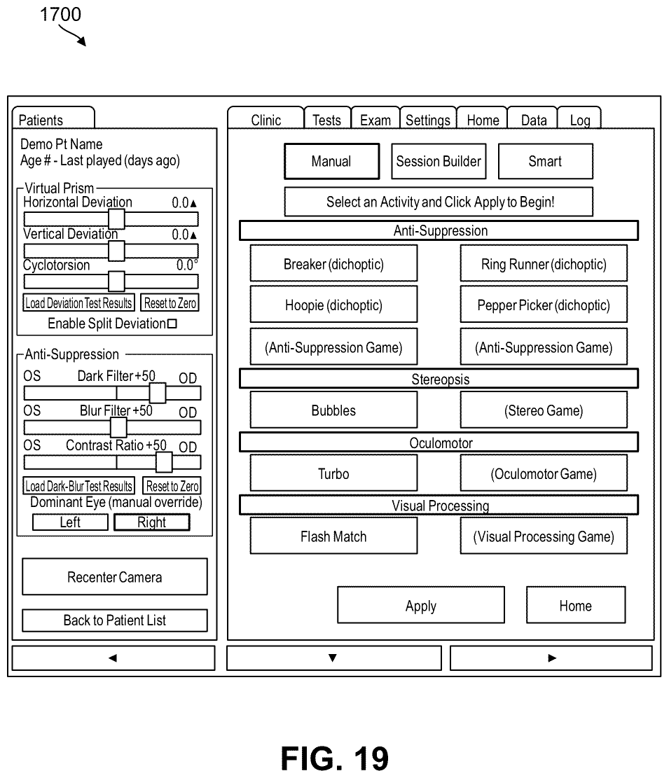

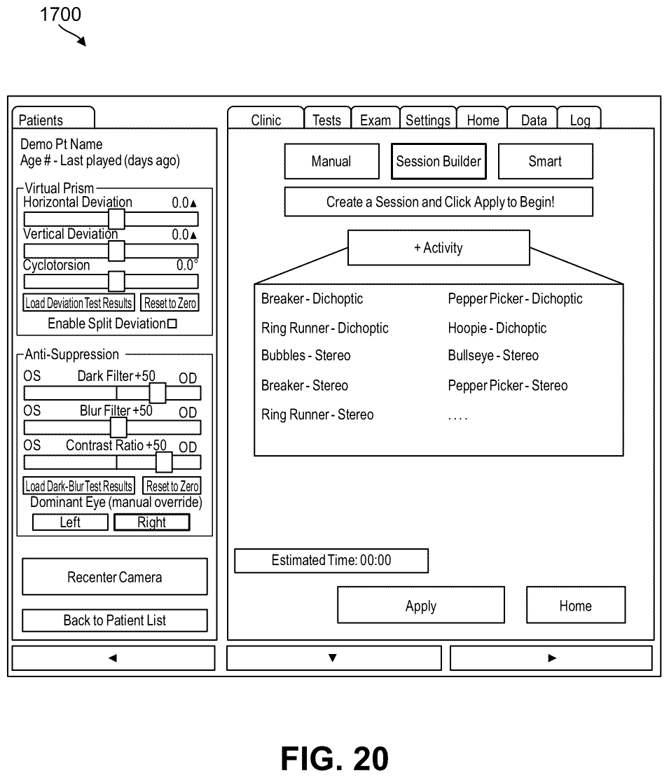

FIGS. 19 and 20 show another embodiment of the exemplary interface 1700. As shown in FIG. 19, the Manual tab 1710 has been selected. Selection of the Manual tab 1710 displays additional selections such as Tracks (e.g. the anti-suppression track, stereopsis track, etc.). As shown in FIG. 20, the Session Builder tab 1715 has been selected, which display additional options such as games.

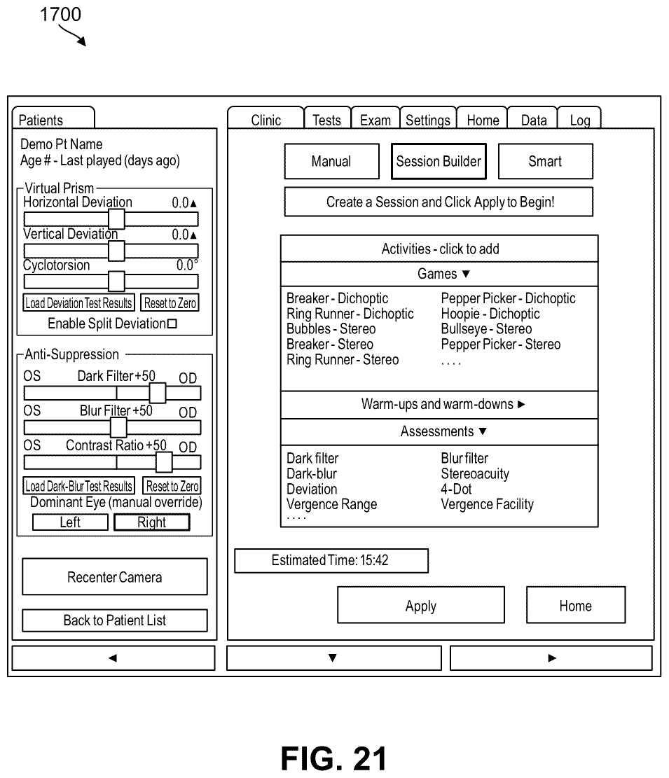

FIG. 21 shows another screen wherein the Session Builder tab 1715 has been selection, and the user may now add activities such as games, assessments, and warm up/warm down exercises. FIG. 21 represents an exemplary implementation, similar to VVH, but including labels to indicate to the clinician what each game's primary play or focus involves. Also shown in FIG. 21 is an estimated time calculator 1725, which includes a best-guess as to the time an average user might take to complete a test.

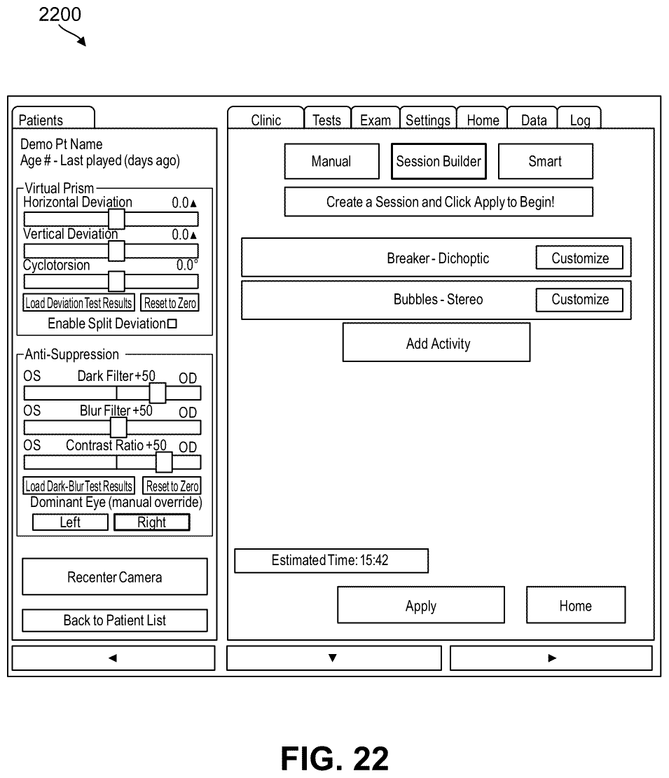

FIG. 22 shows another implementation of an interface 2200 wherein a more organized look is implemented. As shown in FIG. 22, the Session Builder tab 2215 is selected, and additional options for activities are provided, along with an estimated time calculator.

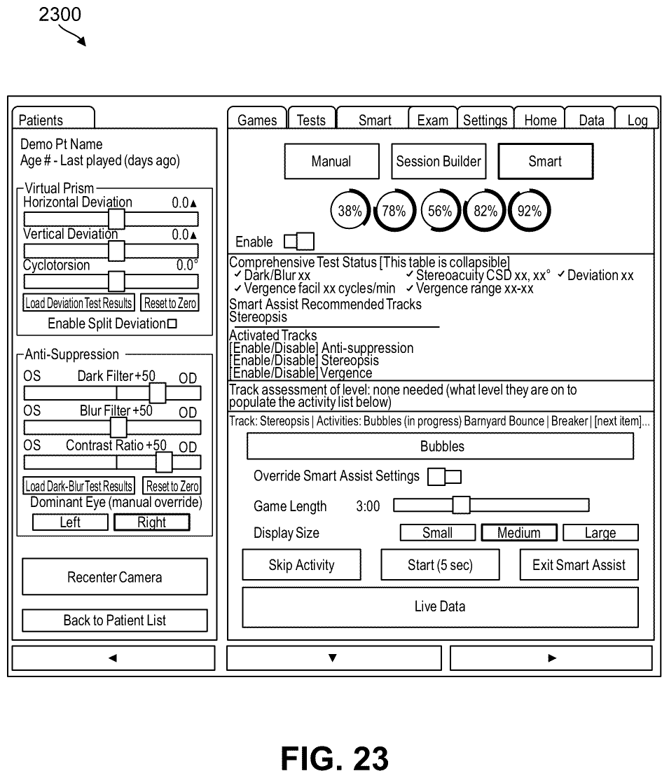

FIG. 23 illustrates another implementation of the interface 2300. As shown in FIG. 23, the clinician has decided to use the Smart tab 2305 option (see top of FIG. 23). Smart assist has been enabled via selection of the Smart tab 2305. Shown in FIG. 23 is the Toggle feature to enable or disable Smart Assist.

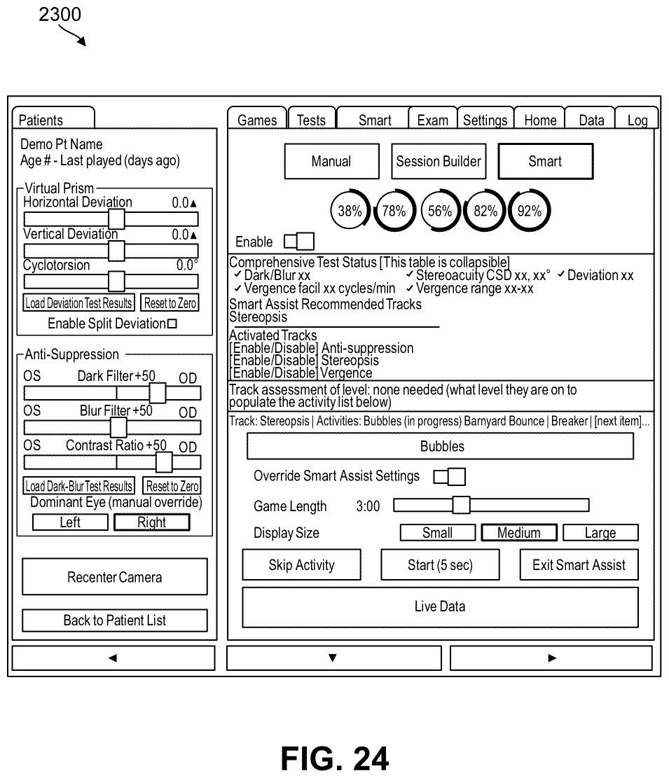

FIG. 24 illustrates the interface 2300. As shown in FIG. 24, the clinician has selected the Override Smart Assist Settings option, enabling the clinician to alter any of the settings (similar to use of the Manual tab 2310). For example, as shown in FIG. 24, the settings are not greyed out any longer in the Bubbles game.

Also shown in FIG. 24, the Start button in this implementation does not have a countdown (i.e. activity manually started). If in-session and the clinician chooses to use the Override Smart Assist Settings option, the button should read Apply.

In some implementations, and example Treatment Plan may include running a full battery of tests in the beginning of treatment (i.e. initial activation of SA). This would then determine the tracks SA would run during treatment. If the clinician de-selects a track(s), this does not affect the testing schedule. The full battery of tests would again be offered at about the seventh session (in clinic) ONLY if the patient is not active in SA with VVA. Alternatively, in VVH, the patient would naturally progress through a series of full-test sessions in the progression of SA).

For example, there may be two scenarios/outcomes of the treatment plan: (1) IF the patient has SA active in VVH, the patient would not need to run the full battery of tests in clinic SA; however, the clinician has the option of running the full test battery in-office if desired, the last test results from VVH SA should be available (results and date); or (2) IF the patient does NOT have SA active in VVH, then the SA program in-clinic would suggest a full test battery.

### Example 5. Treatment Process

An example treatment process is described herein. An example treatment flow-chart is provided in FIG. 7 and described previously.

A Simplified (proposed) Treatment Process is described below.

Selecting Patients for SA

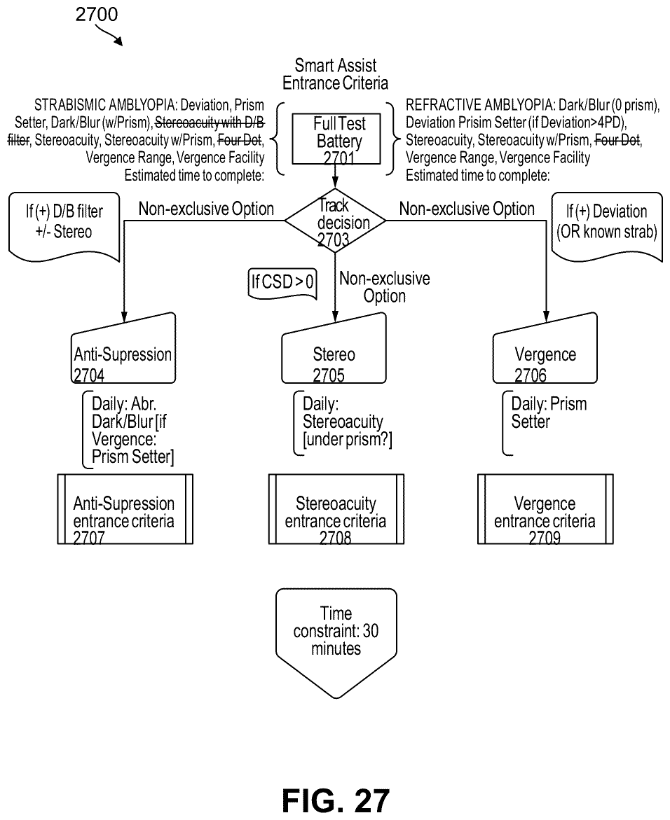

A flow chart for an exemplary selection criteria is shown in FIG. 27, and a general overview of another exemplary is shown in FIG. 5 and described above.

FIG. 27 depicts a flowchart for an example treatment process 2700 for an example vision disorder. In some aspects, the process 2700 is substantially similar to the process 500 described above and illustrated in FIG. 5.

Most patients who are candidates for treatment for their vision disorder are also candidates for treatment with the SA system and methods. In embodiments, before starting SA, the patient should have: (a) at least one clinical exam, with results entered into the VV database, (b) Full Battery of Tests, with result reported to the clinician (e.g. tests done by the patient on their mobile headset in the clinic), and (c) confirmation/editing by the doctor of the SA treatment plan for use of session in the office.

The patient may have a diagnosis to begin with, but SA can also be designed to be fully automatic in terms of providing a sensible, safe treatment for most patients with vergence control difficulties, amblyopia, strabismus, and/or lack of stereopsis. Examples of acceptable conditions that do not preclude treatment include central suppression, eccentric fixation, microstrabismus, and manifest deviations smaller than 30Δ (eso or exo).

Contraindications for use may include anomalous retinal correspondence with a mismatched corresponding location >5Δ (Vivid Vision can be used in patients with anomalous retinal correspondence (ARC) as part of treatment under supervision by a physician, but Smart Assist uses a subjective measure of ocular alignment, so it may not be able to detect or correct the use of binocular vision when the eyes are misaligned due to ARC). Contraindications may also include an inability to fuse after establishing simultaneous perception (e.g. horror fusionis). The Anti-suppression Track can be started without a demonstration of flat fusion or motor fusion, but continued treatment in that track, or starting the Stereopsis Track or Vergence Track, requires demonstration of fusion. At present, the Stereopsis and Vergence Tracks both rely on stereoscopic depth perception for continued progress beyond the earliest levels.

Starting Treatment

In general, treatment starts with a Full Battery test set that evaluates the respective visual skill(s) of a patient. The results from these tests are used to place the patient into one or more Treatment Tracks.

For example, a patient with amblyopia may be given the following exemplary full battery test set: Prism Tuner; Filter (run with prism from Prism Tuner, if any); Stereoacuity (with helping prism, without dark filter); Stereoacuity (without helping prism, without dark filter); FourDot; Vergence Ranges (relative to 200 cm; flat fusion stimuli); Vergence Facility (relative to 200 cm; CSD>10: stereo version. CSD<=10: flat fusion version if they are mature enough to follow instructions).

This example patient may have no need for Virtual Prism (from the Prism Tuner test), which requires a combined Blur and Dark filter from the Filter test, has 900 arcsec of stereoacuity (from Stereoacuity test), suppresses the R eye (from Four Dot test), and has limited vergence ability (Vergence Ranges and Vergence Facility tests). This example patient would therefore benefit from enrollment in 3 tracks: suppression, stereopsis, and vergence tracts.

During Treatment

Patients are assessed using the Full Battery of tests periodically, for example this could be done once every 10 sessions (as described in the Starting Treatment section), and with short daily assessments. These daily short assessments may be thought of as “abbreviated” versions of a test which optimize treatment for the patient through calculated adjustments of activity parameters on a daily basis. Whether at home or in the clinic, SA constructs a suggested session structure. At the end of the initial session (or episode) contents (one or more tracks of treatment), the doctor (or patient if at home) is asked whether to continue the session.

In the aforementioned example, the SA program may suggest a combination of activities within one or more treatment tracks. The clinician may, at any time, manually override or alter available tracks or settings within a (multiple) track(s).

Ending Treatment

Treatment can end when the patient is at Level 10 in all tracks, or fails to show initial improvement, or continued improvement, in at least one track, after 20 sessions (i.e. 4 weeks) of treatment.

This proposed “Endpoint” check system benefits the clinician and patient user. The clinician-user may monitor a patient over time and even remotely. Should a patient fail to progress, the clinician can see this failure as a “flag” to consult with the patient either in-person or remotely. Patient-users may see a lack of progression as an opportunity to self-advocate (if needed) for alterations in treatment or as motivation to more intently participate in the treatment session.

Maintenance

Maintenance can be implemented after “intensive” therapy is completed (i.e. the patient has completed the final level in all specified treatment tracks or the clinician determines that the patient's skillset is adequate). This provides two key items: 1) patient understanding that (as well as motivation to partake in) maintenance therapy that is beneficial to maintain visual skills and 2) an option for clinician oversight that maintains the Doctor-Patient relationship, even with reduced in-person visits.

In a suggested Maintenance Mode, the SA system and associated programing will allow the patient-user more control over games or activities. The SA system should still offer testing at a suggested interval to allow the patient (and overseeing clinician, if applicable) insight into any skill deficiency that may warrant a re-visit to the office.

Parameters that Smart Assist optimizes include the following: Which test to take; Which activity or set of activities to perform and in what order (or that order does not matter); Which data to collect (e.g. Test results, Meta data); Parameters of the activity/test (e.g. Dark/Blur or other filter addition/removal, Virtual Prism addition, alteration, or removal, Size, contrast, shape, color, movement, texture, depth/disparity, luminance, position on retina, position in virtual world); Need to specify inclusion of attachments to any of the tests (e.g. Lenses for near testing), the tests may include the use of attachments, such as plus, minus, bifocal, trifocal, progressive, or prismatic lenses to alter the testing parameters; Image and output rendering for distance, near, or vergence testing; Use of external/peripheral items (e.g. hand-held card or tablet/phone with letters, words, or images or similar for a specific distance) for testing or activity; Use of specified peripherals/feedback devices that interact with the main worn display/device (e.g. biofeedback sensor, electrode, haptic device)); Switching the mode of play within an activity from dichoptic to stereo when the patient is able to perform the task using stereoscopic vision; Blurring only the central part or other specified portion of vision when treating suppression, and peripheral-to-central approach to antisuppression treatment.

Algorithms that Smart Assist uses include the following types of algorithms: Bayesian hyperparameter optimization; AI/ML model-based approaches with lots of data including neural nets, gradient descent, boosted trees, transformers, etc.; Reinforcement learning, both model-based and model-free approaches; Ensembling different models for decision-making; Initial testing; Processing of testing and assessment; Activity and test suggestion OR suggest treatment may not be beneficial; If starting treatment, Real-time monitoring of progression, modification if necessary; Repeat testing and processing and assessment (Improvement or no improvement leads to a suggestion to continue or pause treatment); Update activity and test suggestion; Feedback loop.

Discrete tracks may be used to measure progress independently for separate skills, based on a theory that vision is limited by the weakest of may contributing visual abilities. Assessment at the start of a treatment period, or less often (e.g. once a week), or more often (e.g. frequently during game play), may be used to keep the level of difficulty appropriate for maximum learning to occur by (a) select appropriate activities and (b) selecting appropriate parameters for those activities. Each of the skills is measured by an assessment that targets that tested skill specifically. SA may have the ability to target specific visual skills or work multiple skills at the same time.

Automatic measurement of best prism to use for treatment, with the goal of providing gentle challenge to the patient (not so difficult as to cause fatigue) may be used for the following: Monitoring progress during treatment; Simulation of cover test without eye tracking, using patient's subjective responses (like nonius line alignment), assuming NRC; Simulation of cover test with eye tracking (objective measure), along with patient's subjective response; Use of prism to measure associated phoria vs use of displaced objects in monocular view to measure fixation disparity; Use of several different measures of interocular balance to obtain a profile across distinct mechanisms (suppression of the whole eye when stimuli are different; relative contributions to a cyclopean image when similar images are fused; ability to suppress part of the image by normal observers; all of their corresponding gains in the DSKL model; contribution to apparent luminance, contribution to apparent contrast, percentage of time seen in rivalry stimulus; time to switch in rivalry stimulus; measurement of increment detection threshold in rivalrous or nonrivalrous stimuli); Change of object size within games to overcome suppression (may be monocular or binocular); Change of object side within game to negate or limit aniseikonia; Use of antisuppression checks during game play.

Scaffolding/training wheels may include: General approach of easy-to-hard, building on skills: (1) an implementation changing the Brunswik ratio during game play (a) over the course of a session, (b) from one session to another (Example: ball size in Breaker); and (2) Motion parallax control method, with box moving with the head after maximum excursion reached, (c) it naturally occurs in Barnyard Bounce because a person can do the task using motion parallax, but cannot progress beyond a certain point without stereopsis which is more precise for small disparities.

Hardware and sensors used by Smart Assist include the following: VR; PC; Phone; AR or MR; Eye Tracking (such as to view and record eye and pupil movement, to track accuracy of eye movement during a task, to train eye movement, to alter parameter in activity or test, or to track improvement in eye movement over time); Wearable display; Lenses; Any body movement tracking, including Head Tracking, Facial Tracking, Hand, Foot, or other appendage Tracking, either with or without the use of a controller/sensor or using gesture tracking or haptic feedback; Anything with sound-related sensors or microphone/voice sensor or recognition; Biosensors (e.g. Pupil tracking, Temperature sensor/heart rate sensor/other biofeedback, Skin capacitance, Blood pressure, EEG).

Exemplary hardware for use with the systems and methods discussed herein are disclosed in U.S. patent application Ser. Nos. 14/726,264 and 16/191,324, which are each incorporated by reference herein in their entireties.

Conditions treatable with Smart Assist may include but are not limited to: Anisometropic Amblyopia, Strabismic Amblyopia, Intermittent exotropia, Constant exotropia, Disorders of vergence, Convergence insufficiency, Convergence excess, Divergence insufficiency, Divergence excess, Esotropia (intermittent), Esotropia (caused by far-sightedness), Hypertropia, Cortical suppression, Lack of sensory binocularity needed for flat fusion, Lack of sensory binocularity from, Vergence insufficiency, Accommodative insufficiency, Accommodative infacility, TBI, Anomalous correspondence, Oculomotor, Glaucoma (guidance for treatment, through testing), Low-vision training (eccentric viewing training, scotoma avoidance, specialty prism use [e.g. Pelli prism]), Brain Injury (including concussion, traumatic brain injury, or cerebrovascular accident, Visual field loss, neglect, confusion, scotoma), Lack of or impaired stereoscopic depth perception, Sports vision-related (e.g. Reaction time training, Eye-hand or eye-body coordination).

Visual Skills that can be improved with Smart Assist include the following: skills that are already in the normal range or above normal before treatment, Vergence range, Visual acuity (resolution acuity), Visual acuity (dynamic acuity), Contrast sensitivity, Central-Peripheral awareness, Stereo depth (e.g. fine stereoacuity, course stereo, delta-vergence, for manipulation (e.g. peg-board task), for navigation (e.g. parallel parking, seeing which hallway is real and you'll be able to enter it instead of it's a door with a painting of a hallway on it), for detecting oncoming projectiles, for detecting objects against a background to recognize them, size of spatial integration window), Visual Processing (e.g. Visual discrimination, Visual memory, Spatial relationships, Form constancy, Sequential memory, Visual figure-Ground, Visual closure), Perceptual (e.g. Visual concentration, Visual closure, Visual sequencing, Visual motor integration, Tachistoscope, Visual Search, Visual Scan, Visual Span), Reaction time (e.g. Peripheral, Stereo), Accommodation, Tracking accuracy (e.g. limited by stereo, limited by contrast), Flat fusion, Stereopsis, Awareness of diplopia, and Minimized fixation disparity.

Visual function that may be measured includes the following: Accommodation (Accommodative facility (ability to change accommodation quickly), Accommodation amplitude); Prism vs. vergence (Prism tolerance, Vergence range, Vergence facility, Vergence accuracy); Pupil (individual and right (R) versus left (L)) (Size, Morphology, Reaction to bright/dim light, Reaction to accommodation, Presence or absence of afferent pupillary defect); Color vision; IPD (interpupillary distance); Blinking rate, blink completeness, lid position and symmetry/asymmetry; Head positioning/tilt; Stereoscopic vision; Visual acuity; Contrast sensitivity; Visual field/sensitivity; Scotoma mapping; Central vision analysis/testing for distortion (macular/retinal disorder); Ocular motility/range of motion; Interocular balance; Visual pathway integrity (VEP); Glare sensitivity; Automatic measurement of visual filters, such as dark filter, contrast, and/or blur to use for treatment (within a HMD), with the goal of providing gentle challenge to the patient (not so difficult as to cause fatigue), filters such as dark, contrast, and blur may also be used to monitor progress during treatment; Eye tracking (Monitoring eye positioning during treatment/tests in an HMD; Oculomotor testing such as VOR testing, Oculocephalic testing/Doll's eye reflex testing, Cranial nerve 3-, 4-, 6-evaluation, Saccades, pursuits, vergence, duction, Reading speed, regressions, Speed and accuracy, Nystagmus, Eye position with and without moving body, in any position of gaze, Eye position vs head or body position, Eye position with external stimulus (e.g. caloric), Eye position with or without stimulation of accommodation; Oculomotor training such as Eye-hand coordination measurement using combination of eye tracking and 3D scenes in VR, and Training for reading; and Dynamic tracking instantiations as a method of measuring visual parameters).

Product description from user's point of view

Patient Experience

Smart Assist should be as user friendly as possible for the patient. A series of tests will be repeated, which will guide what settings and activities will be available to a patient. This will give the patient some freedom to choose certain games or activities. The games, activities, tests, and results may be gamified to motivate the patient to continue regular treatment. The patient may be able to view an output showing the automated analyses of data collected by them from games, activities or tests. The patient may view this output using a computer. The output may be transmitted to the patient from the local device that they used to take the test, or from a computing device connected to a central computer by means of the internet. The ability of the patient to view these data (i.e. “see their progress”) may improve engagement as the patient takes a more active role in the treatment process.

Doctor Experience

Smart Assist may be a flexible system or tool that provides help in choosing activities and settings in three different situations: (1) Patient is being seen regularly (anywhere from twice a week to once a month) in the clinic and is also doing home therapy i.e. “Both”; (2) Remote therapy when the patient is not coming into the clinic regularly i.e. “Home only”; and (3) Clinic-only therapy: the patient is not using the system at home (but we hope they will transition onto it) i.e. “Clinic only.”

“Smart Assist is available” may become un-grayed if the battery of tests has been run. Results are blank in the dashboard until done. Also, blank spaces may be included for clinical tests and diagnoses. The system should include date(s) of the tests. The clinician can have full access to data collected by the patient.

As a patient-user progresses through SA, the program can continually and automatically monitor the patient-user's performance in order to label (i.e. “flag”) outputs that are deemed “abnormal” (for example, a patient who has given repeatable stereoacuity values in the past may have a test result that shows a worsening of their stereoacuity skill). Such flags act as a hazard reduction by alerting the clinician to a potentially anomalous or concerning result.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean “based at least in part on” such that an unrecited feature or element is also permissible.

Other implementations than those described herein may be within the scope of the following claims.

1. A computer system for vision assessment and correction, the system comprising:
at least one data processor configured to perform operations comprising:
displaying, using the at least one programmable processor and on a display, a first eye condition assessment or eye condition correction activity of a plurality of eye condition assessments or eye condition correction activities configured to measure a medical disorder or perceptual skill of a user viewing the display;
receiving, by the at least one programmable processor, user input with respect to the first assessment or correction activity, the user input being generated based on input acquired from the user during the eye condition assessment or eye condition correction activity;
determining, by the at least one programmable processor using the received user input, whether a target value of at least one parameter has been reached, wherein the target value of the at least one parameter is indicative of a perception of the first property of the first eye condition assessment or eye condition correction activity by at least one eye of the user;
determining, by the at least one programmable processor based on the target parameter being reached, a current state of the user out of a plurality of possible states within a model of the medical disorder or perceptual skill of the user;
when it is determined that the target value has changed, updating, by the at least one programmable processor, eye conditions assessments and/or eye condition correction activities scheduled for the user by the computing device to reflect the user's current state within the model of the user's medical disorder; and
iteratively performing, by the at least one programmable processor, the displaying, receiving, determining, and updating steps until it is determined that the user's medical condition or perceptual skill has improved to the point where the target value has been reached.

2. The computing system of claim 1, wherein the medical disorder or perceptual skill comprises at least one of anisometropic amblyopia, strabismic amblyopia, intermittent exotropia, constant exotropia, a disorder of vergence, convergence insufficiency, convergence excess, divergence insufficiency, divergence excess, esotropia (intermittent), esotropia, hypertropia, cortical suppression, lack of sensory binocularity needed for flat fusion, lack of sensory binocularity from vergence insufficiency, accommodative insufficiency, accommodative infacility, traumatic brain injury (TBI), anomalous correspondence, oculomotor, glaucoma, low-vision training, brain injury, visual field loss, neglect, confusion, scotoma, lack of or impaired stereoscopic depth perception, a sports vision-related condition.

3. The computing system of claim 1, wherein the plurality of eye condition assessments or eye condition correction activities comprise one or more of: vergence range; visual acuity; contrast sensitivity; central-peripheral awareness; stereo depth; visual processing; perceptual; reaction time; accommodation; tracking accuracy; flat fusion; stereopsis; awareness of diplopia; and minimized fixation disparity.

4. The computing system of claim 1, further comprising a device configured to communicate with the at least one programmable processor, the device comprising a display.

5. The computing system of claim 4, wherein the device comprises one or more of a virtual reality device, a personal computer, a smartphones, an augmented reality device, a mixed reality device, an eye tracking device, a wearable display, a lens, a body movement tracking device, a facial tracking device, an appendage tracking device, a haptic feedback device, an audible sensor, a microphones, and/or a biosensor.

6. A method comprising:
displaying a first eye condition assessment or eye condition correction activity of a plurality of eye condition assessments or eye condition correction activities configured to measure one or more visual skills of a user;
receiving user input with respect to the first assessment or correction activity, the user input being generated based on input acquired from the user during the eye condition assessment or eye condition correction activity;
determining whether a target value of at least one parameter has been reached, wherein the target value of the at least one parameter is indicative of a perception of a first property of the first eye condition assessment or eye condition correction activity by at least one eye of the user;
determining, based on the target parameter being reached, a current state of the user out of a plurality of possible states within a model of a medical disorder or perceptual skill of the user;
updating, when it is determined that the target value has changed, eye conditions assessments or correction activities scheduled for the user to reflect the user's current state within the model of the medical disorder or perceptual skill of the user; and
iteratively performing the displaying, receiving, determining, and updating steps until it is determined that the medical condition or perceptual skill of the user has improved to the point where the target value has been reached.

7. The method of claim 6, wherein the medical disorder or perceptual skill comprises at least one of anisometropic amblyopia, strabismic amblyopia, intermittent exotropia, constant exotropia, a disorder of vergence, convergence insufficiency, convergence excess, divergence insufficiency, divergence excess, esotropia (intermittent), esotropia, hypertropia, cortical suppression, lack of sensory binocularity needed for flat fusion, lack of sensory binocularity from vergence insufficiency, accommodative insufficiency, accommodative infacility, traumatic brain injury (TBI), anomalous correspondence, oculomotor, glaucoma, low-vision training, brain injury, visual field loss, neglect, confusion, scotoma, lack of or impaired stereoscopic depth perception, a sports vision-related condition.

8. The method of claim 6, wherein the plurality of eye condition assessments or eye condition correction activities include one or more of: vergence range; visual acuity; contrast sensitivity; central-peripheral awareness; stereo depth; visual processing; perceptual; reaction time; accommodation; tracking accuracy; flat fusion; stereopsis; awareness of diplopia; and minimized fixation disparity.

9. The method of claim 6, wherein the displaying is done using a device having a display configured to interact with the user.

10. The method of claim 9, wherein the device comprises one or more of a virtual reality device, a personal computer, a smartphones, an augmented reality device, a mixed reality device, an eye tracking device, a wearable display, a lens, a body movement tracking device, a facial tracking device, an appendage tracking device, a haptic feedback device, an audible sensor, a microphones, and/or a biosensor.

11. The method of claim 6, wherein the displaying, the receiving, the determining, the updating, and the iteratively performing are performed using one or more programmable processors.

12. A computer program product comprising a non-transitory computer readable medium storing instructions that, when executed by at least one programmable processor, result in operations comprising:
displaying a first eye condition assessment or eye condition correction activity of a plurality of eye condition assessments or eye condition correction activities configured to measure one or more visual skills of a user;
receiving user input with respect to the first assessment or correction activity, the user input being generated based on input acquired from the user during the eye condition assessment or eye condition correction activity;
determining whether a target value of at least one parameter has been reached, wherein the target value of the at least one parameter is indicative of a perception of a first property of the first eye condition assessment or eye condition correction activity by at least one eye of the user;
determining, based on the target parameter being reached, a current state of the user out of a plurality of possible states within a model of a medical disorder or perceptual skill of the user;
updating, when it is determined that the target value has changed, eye conditions assessments or correction activities scheduled for the user to reflect the user's current state within the model of the medical disorder or perceptual skill of the user; and
iteratively performing the displaying, receiving, determining, and updating steps until it is determined that the medical condition or perceptual skill of the user has improved to the point where the target value has been reached.

13. The computer program product of claim 12, wherein the medical disorder or perceptual skill comprises at least one of anisometropic amblyopia, strabismic amblyopia, Intermittent exotropia, constant exotropia, a disorder of vergence, convergence insufficiency, convergence excess, divergence insufficiency, divergence excess, esotropia (intermittent), esotropia, hypertropia, cortical suppression, lack of sensory binocularity needed for flat fusion, lack of sensory binocularity from vergence insufficiency, accommodative insufficiency, accommodative infacility, traumatic brain injury (TBI), anomalous correspondence, oculomotor, glaucoma, low-vision training, brain injury, visual field loss, neglect, confusion, scotoma, lack of or impaired stereoscopic depth perception, a sports vision-related condition.

14. The computer program product of claim 12, wherein the plurality of eye condition assessments or eye condition correction activities include one or more of: vergence range, visual acuity, contrast sensitivity, central-peripheral awareness, stereo depth; visual processing, perceptual, reaction time, accommodation, tracking accuracy, flat fusion, stereopsis, awareness of diplopia, and minimized fixation disparity.

15. The computer program product of claim 12, wherein the displaying occurs using a device having a display configured to communicate with the user.

16. The computer program product of claim 15, wherein the device comprises one or more of a virtual reality device, a personal computer, a smartphones, an augmented reality device, a mixed reality device, an eye tracking device, a wearable display, a lens, a body movement tracking device, a facial tracking device, an appendage tracking device, a haptic feedback device, an audible sensor, a microphones, and/or a biosensor.
